(19) United States
(12) Patent Application Publication
(10) Pub. No.: US 2026/0165965 A1
(43) Pub. Date: Jun. 18, 2026

(54) STABILIZED TP508 FORMULATIONS

(71) Applicant: Chrysalis BioTherapeutics, Galveston, TX (US)

(72) Inventors: LAURIE E. SOWER, Galveston, TX (US); DARRELL CARNEY, Galveston, TX (US); Steven Pikulin, Galveston, TX (US)

(21) Appl. No.: 18/984,339

(22) Filed: Dec. 17, 2024

(51) Int. Cl. A61K 9/08; A61K 9/00; A61K 38/48; A61K 47/02; A61K 47/22

(52) U.S. Cl. CPC A61K 9/08; A61K 9/0019; A61K 38/4833; A61K 47/02; A61K 47/22

(57) ABSTRACT

Peptide formulations include stable aqueous compositions comprising 50 to 200 mg/mL TP508 or a TP508 derivative and ZnCl₂ or another suitable zinc salt at a concentration of 7 mM to 260 mM at a pH of 5 to 8.5. The peptide formulations can include excipients such as histidine and citrate. These stabilized formulations provide a solution to the problems associated with therapeutic TP508 formulation stability.

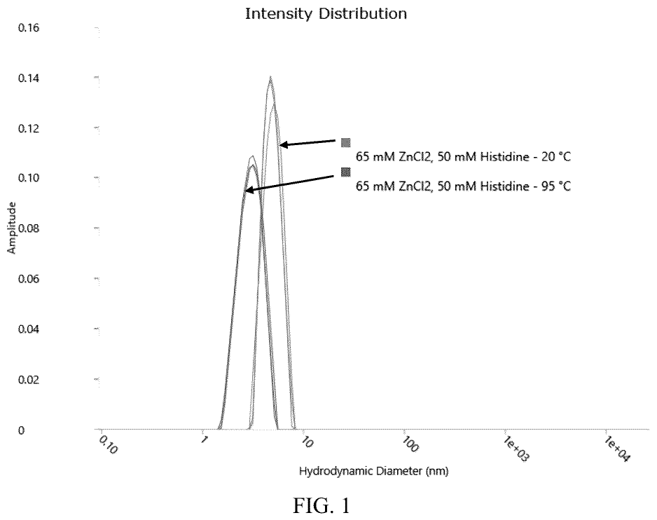

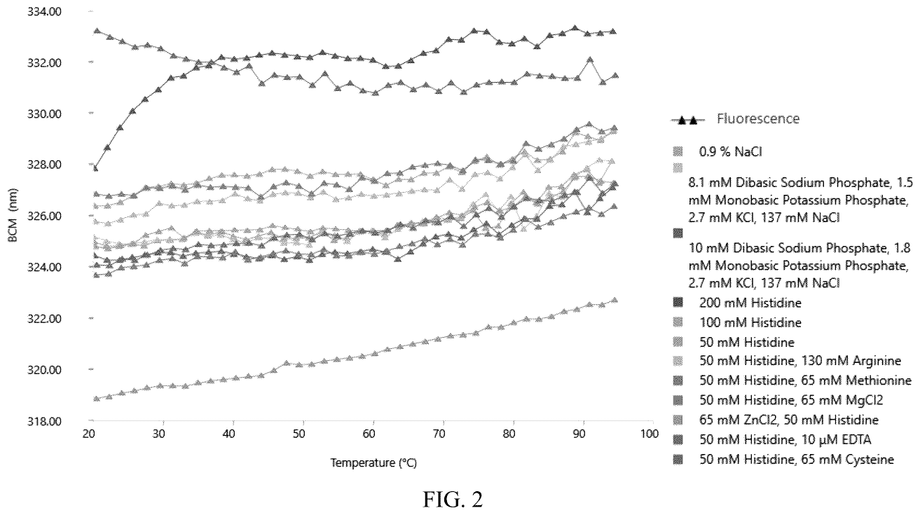

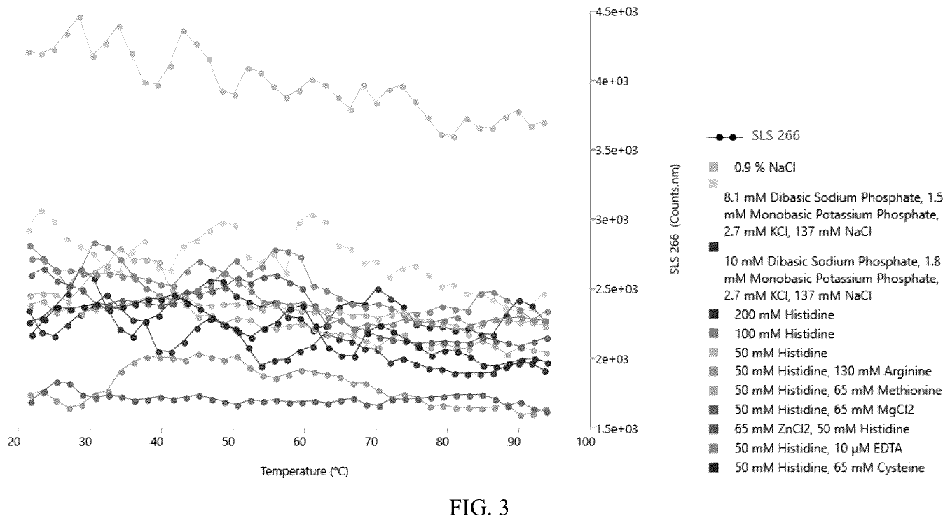

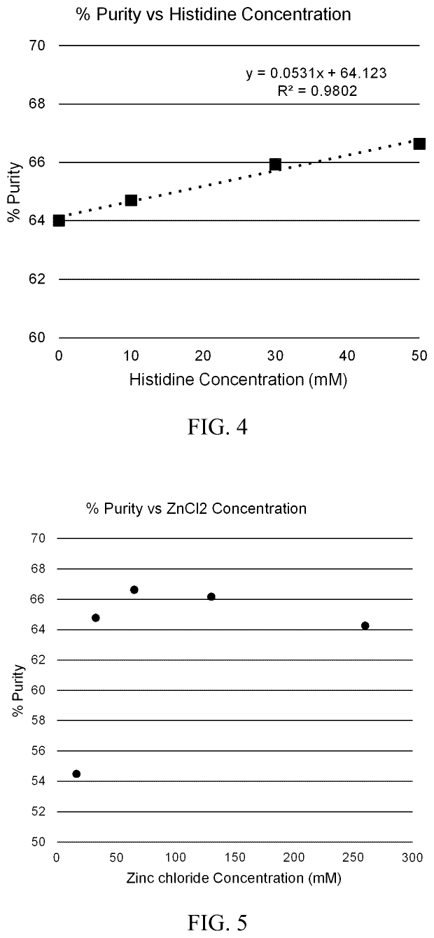

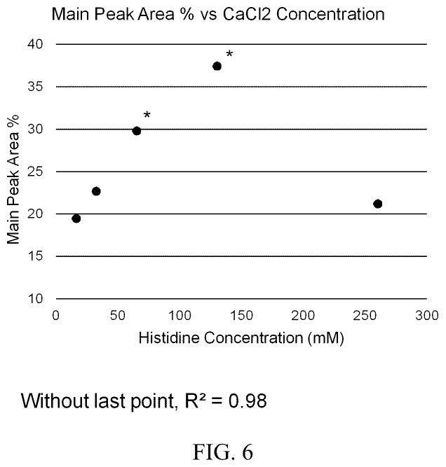

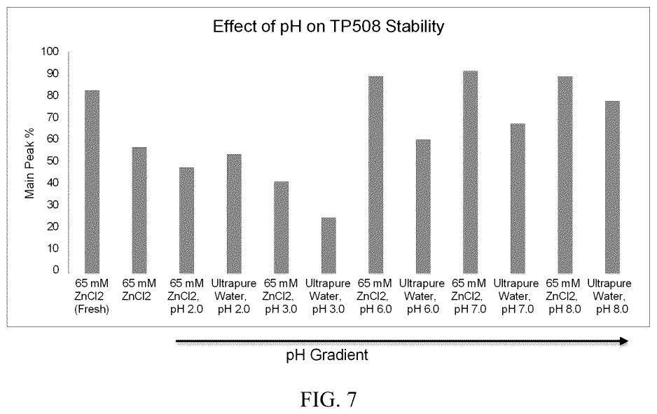

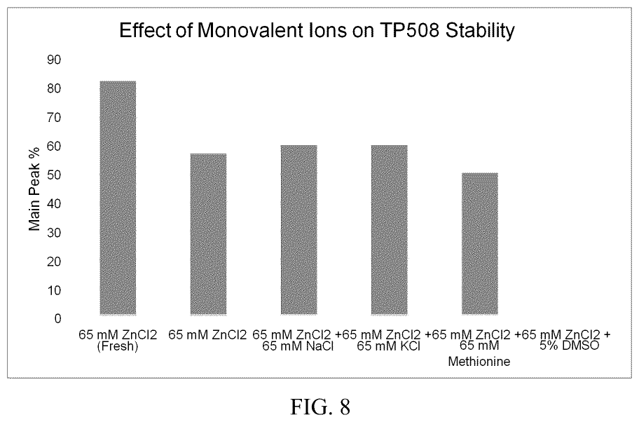

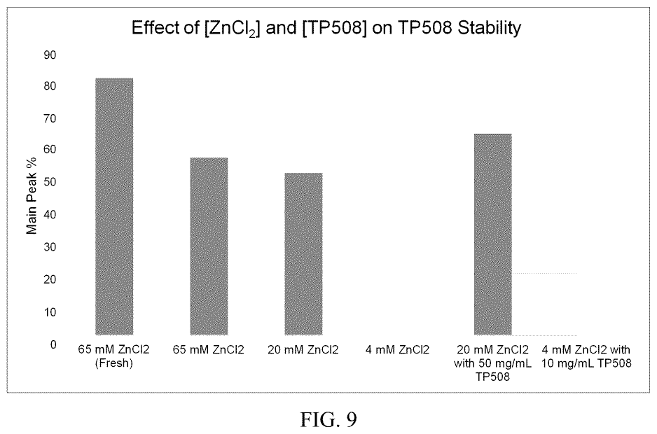

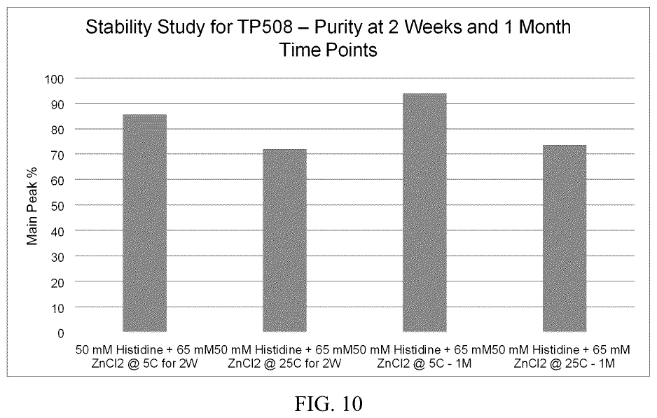

### STATEMENT REGARDING FEDERALLY FUNDED RESEARCH

None.

### REFERENCE TO SEQUENCE LISTING

A sequence listing required by 37 CFR 1.821-1.825 is being submitted electronically with this application. The sequence listing is incorporated herein by reference. The sequence listing that is contained in the file named “CHBI-P0005US” which is 30 KB (as measured in Microsoft Windows®) and was created on Dec. 17, 2024.

### BACKGROUND

TP508 represents a regenerative portion of human thrombin that is released from dissolving blood clots at sites of injury. This portion of thrombin stimulates regeneration of tissue. TP508 has been shown to: (i) stimulate revascularization and restoration of tissue repair in multiple tissues; (ii) protect, recruit, and stimulate proliferation of progenitor stem cells at sites of injury; (iii) modulate immune responses; (iv) restore nitric oxide (NO)-dependent endothelial function; (v) prevent apoptosis; and (vi) mitigate effects of radiation to prevent multiple organ failure and increase survival.

It has been discovered that TP508 prevents TNFα-induced permeability of human pulmonary endothelial cells in vitro. TP508 also counteracts the proinflammatory effects of TNFα on endothelial cells and monocytes, thus serving to reverse pathological inflammatory responses such as those observed in acute respiratory distress syndrome (ARDS) and other inflammatory diseases.

Therapeutic peptides, and peptides in general, consist of multiple amino acid linkages which commonly have reactive sites and three-dimensional spatial configurations. Accordingly, a number of chemical degradation pathways or physical transformations become possible, especially in aqueous media when torsional flexibility is maximized. Degradation pathways for peptides can involve deamidation, racemization, dimerization, hydrolysis, oxidation, beta elimination or disulfide exchange, as well as physical instability related to higher order structure, denaturation, aggregation, precipitation or adsorption. The three most common peptide degradation pathways are aggregation, deamidation, and oxidation with oxidative dimerization being a commonly observed pathway for peptides such as TP508 containing a cysteine residue. For a peptide to remain biologically active, a formulation must preserve intact the conformational integrity of at least a core sequence of the peptide's primary structure while at the same time protecting the peptide's multiple functional groups from degradation. Accordingly, stabilization of peptides in both in vitro and in vivo processes present a special challenge.

This is an especially challenging problem for TP508. In solution, the peptide is only stable in saline solution at 5 degrees C. for 6 to 12 hours. Attempts to formulate TP508 in gels or microsphere preparations have also shown considerable degradation or peptide modification which prohibit their commercial use. Thus, for TP508, it is essential to establish a stabilized form of the molecule to be used as a therapeutic agent.

There remains a need for stabilized TP508 formulations.

### SUMMARY

A solution to the problems associated with therapeutic TP508 formulation stability is provided herein in the form of improved TP508 formulations involving complexation of Zinc (II) with the peptide in the presence or absence of excipients such as histidine and citrate.

Certain embodiments are directed to a stable aqueous composition comprising 50 to 200 mg/mL TP508 or a TP508 derivative and ZnCl₂ or another suitable zinc salt at a concentration of 7 mM to 260 mM at a pH of 5 to 8.5. The composition can include an anti-oxidant. The anti-oxidant can be selected from ascorbic acid, cysteine, methionine, monothioglycerol, sodium thiosulphate, sulfites, BHT, BHA, ascorbyl palmitate, propyl gallate, N-acetyl-L-cysteine (NAC), and Vitamin E. The composition can include one or more sugar. The sugar can be selected from trehalose, glucose, or sucrose. The composition can include a starch. In certain aspects the starch is hydroxyethyl starch (HES). The composition can include a sugar alcohol. In certain aspects the sugar alcohol is mannitol and/or sorbitol. The composition can include a chelator. In certain aspects the chelator is selected from EDTA, tartaric acid, glycerin, and citric acid. The composition can include a preservative. In certain aspects the preservative is selected from benzyl alcohols, methyl parabens, propyl parabens, and mixtures thereof. The composition can include an amino acid or surfactant as an additional excipient(s). In certain aspects an amino acid can be selected from histidine, proline, glycine, or methionine.

Other embodiments of the invention are discussed throughout this application. Any embodiment discussed with respect to one aspect of the invention applies to other aspects of the invention as well and vice versa. Each embodiment described herein is understood to be an embodiment of the invention that is applicable to all aspects of the invention. It is contemplated that any embodiment discussed herein can be implemented with respect to any method or composition of the invention, and vice versa. Furthermore, compositions and kits of the invention can be used to achieve methods of the invention.

As used herein, the term “formulation(s)” means a composition of matter that comprises a combination of at least one active ingredient with one or more other ingredients for one or more particular uses, such as storage, further processing, sale, and/or administration to a subject, such as, for example, administration to a subject of a specific agent in a specific amount, by a specific route, to treat a specific disease. In some embodiments, “formulation” refers to a composition or admixture of a biopharmaceutical and a pharmaceutically acceptable medium that is compatible with the biopharmaceutical that can be administered to humans or animals.

As used herein, the term “excipient” is intended to mean a therapeutically inactive substance. Excipients can be included in a formulation for a wide variety of purposes including, for example, as a diluent, vehicle, buffer, stabilizer, tonicity agent, bulking agent, surfactant, cryoprotectant, lyoprotectant, anti-oxidant, metal ion source, chelating agent and/or preservative. Excipients include, for example, polyols such as sorbitol or mannitol; sugars such as sucrose, lactose or dextrose; polymers such as polyethylene glycol; salts such as ZnCl₂, NaCl, KCl or calcium phosphate; amino acids such as proline, glycine or methionine; surfactants; metal ions; buffer salts such as glutamate, acetate or aspartate; preservatives; and polypeptides such as human serum albumin, as well as saline and water. Excipients can comprise sugars, for example sugar alcohols, reducing sugars, non-reducing sugars and sugar acids. Excipients are well known in the art and can be found described in, for example, Wang, Int. J. Pharm. 185:129-88 (1999) and Wang, Int. J. Pharm. 203:1-60 (2000).

“Chemical stability,” when referring to a therapeutic agent, refers to an acceptable percentage of degradation products produced by chemical pathways involving cleavage or formation of covalent bonds. These include oxidation, hydrolysis, fragmentation, dimerization and/or other chemical degradation pathways involving covalent bond cleavage, formation and/or rearrangement. It is important to note that as described herein, chemical degradation is not restricted to breakdown of the molecular structure of the therapeutic agent; it may also involve covalent bonding of a species to the therapeutic agent molecule (such as in dimerization), resulting in a degradant with molecular weight larger than that of the therapeutic agent. A formulation of the type described herein may be considered chemically stable if no more than about 20% degradation products are formed after at least 6 months of storage at refrigerated storage or subzero storage; or storage of the product at accelerated conditions (25° C./60% relative humidity) for one month, two months, or three months. In some embodiments, a chemically stable formulation has less than 30%, less than 25%, less than 20%, less than 15%, less than 10%, less than 5%, less than 4%, less than 3%, less than 2%, or less than 1% degradation products formed after an extended period of storage (1, 2, 3, 4, 5, 6, 7, 8, 9, 10, 11, 12 or more weeks or months) at the intended storage temperature (−20, −10, −2, 0, 2, 8, 10, 20, 30, 40, 50, degrees C.) of the product.

“Physical stability,” when referring to a therapeutic agent, refers to an acceptable percentage of aggregates being formed. As used herein, aggregates refer specifically to larger order structures formed via hydrophobic effects, van der Waals interactions, π-π stacking, hydrogen bonding, and/or other non-covalent interactions. These are in contrast to dimers and higher order oligomers which form through covalent bonding and are considered as part of chemical stability. Aggregate formations may be reversible or irreversible, with their stability depending upon many factors such as amino acid sequence, peptide concentration, spatial configuration and other variables. Aggregate formation is generally unintended, whereas some embodiments are directed to an intended dimer which would be excluded from the unintended aggregates given the circumstances of an intentional dimer. In particular, a formulation is considered physically stable if no more than about 15% aggregates are formed after at least 6 months of storage at refrigerated storage or subzero storage; or storage of the product at 25° C./60% relative humidity for one month, two months, or three months. In some embodiments, a physically stable formulation has less than 15%, less than 10%, less than 5%, less than 4%, less than 3%, less than 2%, or less than 1% aggregates formed after an extended period of storage at the intended storage temperature of the product.

“Stable formulation” refers to a formulation where at least about 65% or more of the therapeutic agents (e.g., peptides or salts thereof) remain chemically and physically stable after at least one month of storage at room temperature, or up to at least one year of storage at refrigerated or subzero temperatures. Particularly preferred formulations are those in which at least about 70%, 75%, 80%, 85%, 90%, 91%, 92%, 93%, 94%, 95%, 96%, 97%, 98%, or 99% chemically and physically stable therapeutic agent remains under these storage conditions. Especially preferred stable formulations are those which do not exhibit degradation after sterilizing (e.g., gamma or electron beam (aka beta)) irradiation.

As used herein, “formulation for administration” refers to the administration of therapeutic agents (e.g., peptides) via a route other than the alimentary canal-any administration that is not by way of the digestive tract—for example, intravenous infusion, intranasal administration, buccal administration, transdermal administration, or parenteral injection under or through one or more layers of skin or mucus membranes of an animal, such as a human. Standard parenteral injections are given into the subcutaneous, intramuscular, or intradermal tissues of an animal, including humans. These deep locations are targeted because the tissue expands more easily relative to shallow dermal sites to accommodate injection volumes required to deliver most therapeutic agents, e.g., 0.1 to 3.0 cc (mL).

The term “about” or “approximately” or “substantially unchanged” are defined as being close to as understood by one of ordinary skill in the art, and in one non-limiting embodiment the terms are defined to be within 10%, preferably within 5%, more preferably within 1%, and most preferably within 0.5%.

“Pharmaceutically acceptable” ingredient, excipient or component is one that is suitable for use with humans and/or animals without undue adverse side effects (such as toxicity, irritation and allergic response) commensurate with a reasonable benefit/risk ratio.

“Pharmaceutically acceptable carrier” means a pharmaceutically acceptable solvent, suspending agent, or vehicle for delivering a drug compound of the present invention to a mammal such as a human.

As used herein, a “mineral acid” is an acid that is derived from one or more inorganic compounds. Accordingly, mineral acids may also be referred to as “inorganic acids.” Mineral acids may be monoprotic or polyprotic (e.g. diprotic, triprotic, etc.). Non-limiting examples of mineral acids include hydrochloric acid (HCl), nitric acid (HNO₃), sulfuric acid (H₂SO₄), and phosphoric acid (H₃PO₄).

As used herein, a “mineral base” (which may be equally and alternatively referred to as an “inorganic base”) is a base that is derived from one or more inorganic compounds. Many, but not all, inorganic bases are generally classified as “strong bases,” and non-limiting examples of inorganic bases include sodium hydroxide (NaOH), potassium hydroxide (KOH), magnesium hydroxide (Mg(OH)₂) and calcium hydroxide (Ca(OH)₂).

As used herein, an “organic acid” is an organic compound with acidic properties (i.e., can function as a proton source). Carboxylic acids, such as acetic acid or citric acid, are one example of organic acids. Other known examples of organic acids include, but are not limited to, alcohols, thiols, enols, phenols, and sulfonic acids. Organic acids may be monoprotic or polyprotic (e.g., diprotic, triprotic, etc.).

As used herein, an “organic base” is an organic compound with basic properties (i.e., it can function as a proton acceptor/sink). Many, but not all, organic bases contain nitrogen atoms (e.g., amines), and non-limiting examples of organic bases include amino acids (e.g., histidine, arginine, lysine), pyridine, imidazole and tromethamine. Organic bases may accept one or more protons per molecule.

As used herein, a “co-formulation” is a formulation that contains two or more therapeutic agents in a formulation. The therapeutic agents may belong to the same class (for example, a co-formulation comprising two or more therapeutic peptides), or the therapeutic agents may belong to different classes (for example a co-formulation comprising one or more therapeutic small molecules and one or more therapeutic peptide molecules).

The term “therapeutic agent” encompasses peptide compounds together with pharmaceutically acceptable salts thereof. Useful salts are known to those skilled in the art and include salts with inorganic acids, organic acids, inorganic bases, or organic bases. Therapeutic agents useful in the present invention are those peptide compounds that affects a desired, beneficial, and often pharmacological, effect upon administration to a human or an animal, whether alone or in combination with other pharmaceutical excipients or inert ingredients.

The terms “peptide,” “polypeptide” and/or “peptide compound” refer to compounds of 5 amino acid residues up to about 80 amino acid residues (e.g., TP508 at 23 amino acid residues) bound together by amide (CONH) linkages. Analogs, derivatives, agonists, antagonists and pharmaceutically acceptable salts of any of the peptide compounds disclosed here are included in these terms. The terms also include peptides and/or peptide compounds that have D-amino acids, modified, derivatized or non-naturally occurring amino acids in the D- or L-configuration and/or peptomimetic units as part of their structure.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, a person skilled in the art (“POSITA”) will understand that the following description has broad application, and the discussion of any embodiment is meant only to be an example of that embodiment, and not intended to imply that the scope of the disclosure, including the claims, is limited to that embodiment.

It is well-known that some proteins and peptides can bind or complex to metals, which can affect their chemical and/or biological properties including thermal stability, catalysis of in vitro or in vivo reaction processes, therapeutic activity or toxicity. In aqueous media, these effects often occur in concert with solution pH and/or the pH buffer anion. For example, the stability of oxytocin (a nonapeptide with cysteine residues) in aqueous formulations was improved in the presence of 5 and 10 mM citrate buffer in combination with at least 2 mM CaCl₂, MgCl₂, or ZnCl₂ and depended on the divalent metal ion concentration (Avanti, AAPS J. 13 (2): 284-290 (2011)). Isothermal titration calorimetric measurements were predictive for the stabilization effects observed during the stability study. Formulations in citrate buffer that had an improved stability displayed a strong interaction between oxytocin and Ca, Mg, or Zn ions, while formulations in acetate buffer did not. Similarly, the DNA binding properties of H4Sp1 with different divalent metal ions demonstrated that only Zn(II) reconstituted H4Sp1 bound strongly to the DNA GC-box (Hori, ICR Ann. Rep. Vol. 7 (2000)). These and other examples provide a general strategy for stabilization of TP508 in aqueous formulation to achieve a more desirable stability/storage profile. However, there are no established formulations or protocols that universally stabilize peptides, and none that have been shown to stabilize TP508. Although every peptide has unique features that dictate how one might best provide stabilization, these and other examples provide a strategy by which we have established a practical formulation for stabilization of TP508 in aqueous formulation to achieve a more desirable stability/storage profile.

### I. PEPTIDE FORMULATIONS

In one aspect, the present invention provides a stable formulation for storage, shipping, and/or administration. Advantageously, once prepared, the formulation is stable for extended periods of time, is ready for use without the need for reconstitution, and is functional over a range of temperatures. In some embodiments, the formulations of the present invention increase the physical stability and/or chemical stability of the peptide or peptides of the formulation, for example, by preventing or decreasing the formation of aggregates of the peptide or peptides, or by preventing or decreasing the degradation of the peptide or peptides. In some embodiments, the formulation comprises: (a) a peptide or a salt thereof, and (b) an aqueous solution.

### A. TP508 Peptide or TP508 Derivatives

The stable formulations of the present invention comprise one, two, three, four, or more peptides or salts, analogs, and/or mixtures thereof. Peptides (as well as salts thereof) suitable for use in the formulations of the present invention include, but are not limited to, TP508 or a TP508 derivative, and mixtures thereof. In a preferred embodiment, the peptide is TP508 (SEQ ID NO:1), or a salt thereof. In some embodiments, the formulation comprises two peptides with the first peptide being TP508 (SEQ ID NO:1).

Thrombin peptide (TP) derivatives (also: “thrombin derivative peptides”) are analogs of thrombin that have an amino acid sequence derived at least in part from that of thrombin and are active at the non-proteolytically activated thrombin receptor (NPAR). Thrombin peptide derivatives can include, for example, peptides that are produced by recombinant DNA methods, peptides produced by enzymatic digestion of thrombin, and peptides produced synthetically, which can comprise amino acid substitutions compared to thrombin and/or modified amino acids, especially at one or both termini.

Thrombin peptide derivatives of the present invention include thrombin derivative peptides described in U.S. Pat. Nos. 5,352,664 and 5,500,412, each of which is incorporated herein by reference in their entirety. In one embodiment, the thrombin peptide derivatives of the present invention is a thrombin peptide derivative or a physiologically functional equivalent, i.e., a polypeptide with no more than about fifty amino acids, preferably no more than about thirty amino acids and having sufficient homology to the fragment of human thrombin corresponding to thrombin amino acids 508-530 (AGYKPDEGKRGDACEGDSGGPFV (SEQ ID NO:1, TP508)) that the polypeptide activates NPAR.

In another embodiment, the thrombin peptide derivative of the present invention is a thrombin peptide derivative comprising a moiety represented by Structural Formula (I) Asp-Ala-R, where R is a serine esterase conserved domain. Serine esterases, e.g., trypsin, thrombin, chymotrypsin and the like, have a region that is highly conserved. “Serine esterase conserved domain” refers to a polypeptide having the amino acid sequence of one of these conserved regions or is sufficiently homologous to one of these conserved regions such that the thrombin peptide derivative retains NPAR activating ability.

A physiologically functional equivalent of a thrombin derivative encompasses molecules which differ from thrombin derivatives in aspects which do not affect the function of the thrombin receptor binding domain or the serine esterase conserved amino acid sequence. Such aspects may include, but are not limited to, conservative amino acid substitutions and modifications, for example, amidation of the carboxyl terminus, acetylation of the amino terminus, conjugation of the polypeptide to a physiologically inert carrier molecule, or sequence alterations in accordance with the serine esterase conserved sequences.

In one embodiment, the serine esterase conserved sequence comprises the amino acid sequence of SEQ ID NO:2 (CEGDSGGPFV) or a C-terminal truncated fragment of a polypeptide having the amino acid sequence of SEQ ID NO:2. It is understood, however, that zero, one, two or three amino acids in the serine esterase conserved sequence can differ from the corresponding amino acid in SEQ ID NO:2. Preferably, the amino acids in the serine esterase conserved sequence which differ from the corresponding amino acid in SEQ ID NO:2 are conservative substitutions as defined below, and are more preferably highly conservative substitutions. A “C-terminal truncated fragment” refers to a fragment remaining after removing an amino acid or block of amino acids from the C-terminus, said fragment having at least six and more preferably at least nine amino acids.

In another embodiment, the serine esterase conserved sequence comprises the amino acid sequence of SEQ ID NO:3 (CX1GDSGGPX2V; X1 is Glu or Gln and X2 is Phe, Met, Leu, His or Val) or a C-terminal truncated fragment thereof having at least six amino acids, preferably at least nine amino acids. In a preferred embodiment, the thrombin peptide derivative comprises a serine esterase conserved sequence and a polypeptide having a more specific thrombin amino acid sequence Arg-Gly-Asp-Ala. One example of a thrombin peptide derivative of this type comprises RGDACX1GDSGGPX2V (SEQ ID NO:4). X1 and X2 are as defined above. The thrombin peptide derivative can comprise the amino acid sequence of AGYKPDEGKRGDACEGDSGGPFV (SEQ ID NO: 5) or an N-terminal truncated fragment thereof, provided that zero, one, two or three amino acids at positions 1-9 in the thrombin peptide derivative differ from the amino acid at the corresponding position of SEQ ID NO:4. Preferably, the amino acid residues in the thrombin peptide derivative which differ from the corresponding amino acid residues in SEQ ID NO:4 are conservative substitutions as defined below, and are more preferably highly conservative substitutions. An “N-terminal truncated fragment” refers to a fragment remaining after removing an amino acid or block of amino acids from the N-terminus, preferably a block of no more than six amino acids, more preferably a block of no more than three amino acids.

Optionally, the thrombin peptide derivatives described herein can be amidated at the C-terminus and/or acylated at the N-terminus. In a specific embodiment, the thrombin peptide derivatives comprise a C-terminal amide and optionally comprise an acylated N-terminus, wherein said C-terminal amide is represented by —C(O)NRaRb, wherein Ra and Rb are independently hydrogen, a C1-C10 substituted or unsubstituted aliphatic group, or Ra and Rb, taken together with the nitrogen to which they are bonded, form a C1-C10 non-aromatic heterocyclic group, and said N-terminal acyl group is represented by RcC(O)—, wherein Rc is hydrogen, a C1-C10 substituted or unsubstituted aromatic group, or a C1-C10 substituted or unsubstituted aromatic group. In another embodiment, the N-terminus of the thrombin peptide derivative is free (i.e., unsubstituted) and the C-terminus is free (i.e., unsubstituted) or amidated, preferably as a carboxamide (i.e., —C(O)NH₂). In one embodiment, the thrombin peptide derivative comprises the following amino acid sequence: AGYKPDEGKRGDACEGDSGGPFV (SEQ ID NO:5). In another embodiment, the thrombin peptide derivative comprises the amino sequence of RGDACEGDSGGPFV (SEQ ID NO: 6). Alternatively, the thrombin peptide derivative comprises the amino acid sequence of DNMFCAGYKPDEGKRGDACEGDSGGPFVMKSPF (SEQ ID NO:7). The thrombin peptide derivatives can optionally be amidated at the C-terminus and/or acylated at the N-terminus. Preferably, the N-terminus is free (i.e., unsubstituted) and the C-terminus is free (i.e., unsubstituted) or amidated, preferably a carboxamide (i.e., —C(O)NH₂). It is understood, however, that zero, one, two or three amino acids at positions 1-9 and 14-23 in the thrombin peptide derivative can differ from the corresponding amino acids.

Preferably, the amino acids in the thrombin peptide derivative which differ from the corresponding amino acids are conservative substitutions as defined below and are more preferably highly conservative substitutions. Alternatively, an N-terminal truncated fragment of the thrombin peptide derivative having at least fourteen amino acids or a C-terminal truncated fragment of the thrombin peptide derivative having at least eighteen amino acids can be used in the methods of the present invention.

A “C-terminal truncated fragment” refers to a fragment remaining after removing an amino acid or block of amino acids from the carboxy- or C-terminus. An “N-terminal truncated fragment” refers to a fragment remaining after removing an amino acid or block of amino acids from the amino- or N-terminus. It is to be understood that the terms “C-terminal truncated fragment” and “N-terminal truncated fragment” encompass acylation at the N-terminus and/or amidation at the C-terminus, as described above.

A preferred thrombin peptide derivative for use in the disclosed method comprises the amino acid sequence AGYKPDEGKRGDACX1GDSGGPX2V (SEQ ID NO:8). Another preferred thrombin peptide derivative for use in the disclosed method comprises the amino acid sequence of DNMFCAGYKPDEGKRGDACX1GDSGGPX2VMKSPF (SEQ ID NO:9), wherein X1 is Glu or Gln; X2 is Phe, Met, Leu, His or Val. The thrombin peptide derivatives can optionally comprise a C-terminal amide and/or acylated N-terminus, as defined above. Preferably, the N-terminus is free (i.e., unsubstituted) and the C-terminus is free (i.e., unsubstituted) or amidated, preferably as a carboxamide (i.e., —C(O)NH₂). Alternatively, N-terminal truncated fragments of these preferred thrombin peptide derivatives, the N-terminal truncated fragments having at least fourteen amino acids, or C-terminal truncated fragments of these preferred thrombin peptide derivatives, the C-terminal truncated fragments having at least eighteen amino acids, can also be used in the disclosed method.

TP508 is an example of a thrombin peptide derivative and is 23 amino acid residues, wherein the N-terminal amino acid residue Ala is unsubstituted and the COOH of the C-terminal amino acid Val is modified to an amide represented by —C(O)NH₂. Another example of a thrombin peptide derivative comprises the amino acid sequence where both N- and C-termini are unsubstituted (“desamide TP508”). Other examples of thrombin peptide derivatives which can be used in the disclosed methods and compositions include N-terminal truncated fragments of TP508 (or desamide TP508), the N-terminal truncated fragments having at least fourteen amino acids, or C-terminal truncated fragments of TP508 (or desamide TP508), the C-terminal truncated fragments having at least eighteen amino acids.

As used herein, a “conservative substitution” in a polypeptide or peptide is the replacement of an amino acid with another amino acid that has the same net electronic charge and approximately the same size and shape. Amino acids with aliphatic or substituted aliphatic amino acid side chains have approximately the same size when the total number of carbon and heteroatoms in their side chains differs by no more than about four. They have approximately the same shape when the number of branches in their side chains differs by no more than one. Amino acids with phenyl or substituted phenyl groups in their side chains are considered to have about the same size and shape. Listed below are five groups of amino acids. Replacing an amino acid in a polypeptide or peptide with another amino acid from the same group results in a conservative substitution:
Group I: glycine, alanine, valine, leucine, isoleucine, serine, threonine, cysteine, and non-naturally occurring amino acids with C1-C4 aliphatic or C1-C4 hydroxyl substituted aliphatic side chains (straight chained or monobranched).
Group II: glutamic acid, aspartic acid and non-naturally occurring amino acids with carboxylic acid substituted C1-C4 aliphatic side chains (unbranched or one branch point).
Group III: lysine, ornithine, arginine and non-naturally occurring amino acids with amine or guanidino substituted C1-C4 aliphatic side chains (unbranched or one branch point).
Group IV: glutamine, asparagine and non-naturally occurring amino acids with amide substituted C1-C4 aliphatic side chains (unbranched or one branch point).
Group V: phenylalanine, phenylglycine, tyrosine and tryptophan.

As used herein, a “highly conservative substitution” in a polypeptide is the replacement of an amino acid with another amino acid that has the same functional group in the side chain and nearly the same size and shape. Amino acids with aliphatic or substituted aliphatic amino acid side chains have nearly the same size when the total number of carbon and heteroatoms in their side chains differs by no more than two. They have nearly the same shape when they have the same number of branches in their side chains. Examples of highly conservative substitutions include valine for leucine, threonine for serine, aspartic acid for glutamic acid and phenylglycine for phenylalanine. Examples of substitutions which are not highly conservative include alanine for valine, alanine for serine and aspartic acid for serine.

### B. Modified Thrombin Peptide Derivatives

In one embodiment of the invention, the thrombin peptide derivatives are modified relative to the thrombin peptide derivatives described above, wherein cysteine residues of thrombin peptide derivatives are replaced with amino acids having similar size and charge properties to minimize dimerization of the peptides. Examples of suitable amino acids include alanine, glycine, serine, or an S′-protected cysteine. Preferably, cysteine is replaced with alanine. The modified thrombin peptide derivatives have about the same biological activity as the unmodified thrombin peptide derivatives. See Publication No. US 2005/0158301 A1, which is hereby incorporated by reference.

It will be understood that the modified thrombin peptide derivatives disclosed herein can optionally comprise C-terminal amides and/or N-terminal acyl groups, as described above. In one aspect, the N-terminus of a thrombin peptide derivative is free (i.e., unsubstituted) and the C-terminus is free (i.e., unsubstituted) or amidated, for example as a carboxamide (i.e., —C(O)NH₂).

In one embodiment, the modified thrombin peptide derivative comprises a polypeptide or peptide having the amino acid sequence of RGDAX3X1GDSGGPX2V (SEQ ID NO:10), or a C-terminal truncated fragment thereof having at least six amino acids. More specifically, the thrombin peptide derivative comprises the amino acid sequence of AGYKPDEGKRGDAX3EGDSGGPFV (SEQ ID NO:11) or a fragment thereof comprising amino acids 10-18 of peptide. Even more specifically, the thrombin peptide derivative comprises the amino acid sequence AGYKPDEGKRGDAX3X1GDSGGPX2V (SEQ ID NO:12), or a fragment thereof comprising amino acids 10-18 of sequence. X3 is alanine, glycine, serine or an S-protected cysteine. X1 is Glu or Gln and X2 is Phe, Met, Leu, His or Val. Preferably X1 is Glu, X2 is Phe, and X3 is alanine. One example of a thrombin peptide derivative of this type is a polypeptide having the amino acid sequence AGYKPDEGKRGDAAEGDSGGPFV (SEQ ID NO:13). A further example of a thrombin peptide derivative of this type is the polypeptide H-AGYKPDEGKRGDAAEGDSGGPFV-NH₂ (SEQ ID NO:14). Another example of a thrombin peptide derivative of this type is the polypeptide H-AGYKPDEGKRGDASEGDSGGPFV-NH₂ (SEQ ID NO:15). Zero, one, two or three amino acids in the thrombin peptide derivative differ from the amino acid at the corresponding position of the sequences. Preferably, the difference is conservative as defined herein.

In another embodiment, the thrombin peptide derivative comprises a polypeptide having the amino acid sequence DNMFX4AGYKPDEGKRGDAX5EGDSGGPFVMKSPF (SEQ ID NO: 16), or a fragment thereof comprising amino acids 6-28. More preferably, the thrombin peptide derivative comprises a polypeptide having the amino acid sequence DNMFX4AGYKPDEGKRGDAX5X1GDASGGPX2VMKSPF (SEQ ID NO:17), or a fragment thereof comprising amino acids 6-28. X5 and X4 are independently alanine, glycine, serine or an S-protected cysteine. X1 is Glu or Gln and X2 is Phe, Met, Leu, His or Val. Preferably X1 is Glu, X2 is Phe, and X5 and X4 are alanine. One example of a thrombin peptide derivative of this type is a polypeptide comprising the amino acid sequence DNMFAAGYKPDEGKRGDAAEGDSGGPFVMDSPF (SEQ ID NO:18). A further example of a thrombin peptide derivative of this type is the polypeptide COOH-DNMFAAGYKPDEGKRGDAAEGDSGGPFVMKSPF-NH₂ (SEQ ID NO:19). Zero, one, two or three amino acids in the thrombin peptide derivative can differ from the amino acid at the corresponding position of the sequences. X5 and X4 are independently alanine, glycine, serine or an S-protected cysteine. Preferably, the difference is conservative as in conservative substitutions of the thrombin peptide derivatives.

An “S-protected cysteine” is a cysteine residue in which the reactivity of the thiol moiety, —SH, is blocked with a protecting group. Suitable protecting groups are known in the art and are disclosed, for example, in Greene and Wuts, Protective Groups in Organic Synthesis, 3rd Edition, John Wiley & Sons, (1999), pp. 454-493. A free thiol can be protected as a thioether, a thioester, or can be oxidized to an unsymmetrical disulfide. In certain aspects the thiol is protected as a thioether. Suitable thioethers include, but are not limited to, S-alkyl thioethers (e.g., C1-C5 alkyl), and S-benzyl thioethers (e.g. cysteine-S-S-i-Bu). Preferably the protective group is an alkyl thioether. More preferably, the S-protected cysteine is an S-methyl cysteine. Alternatively, the protecting group can be: (1) a cysteine or a cysteine-containing peptide (the “protecting peptide”) attached to the cysteine thiol group of the thrombin peptide derivative by a disulfide bond; or (2) an amino acid or peptide (“protecting peptide”) attached by a thioamide bond between the cysteine thiol group of the thrombin peptide derivative and a carboxylic acid in the protecting peptide (e.g., at the C-terminus or side chain of aspartic acid or glutamic acid). The protecting peptide can be physiologically inert (e.g., a polyglycine or polyalanine of no more than about fifty amino acids optionally interrupted by a cysteine), or can have a desirable biological activity.

### C. Thrombin Peptide Derivative Dimers

In some aspects of the present invention, the thrombin peptide derivatives of the methods are thrombin peptide derivative dimers. See Publication No. US 2005/0153893, which is hereby incorporated by reference. The dimers essentially do not revert to monomers and still have about the same biological activity as the thrombin peptide derivative monomers described above. A “thrombin peptide derivative dimer” is a molecule comprising two thrombin peptide derivatives linked by a covalent bond, preferably a disulfide bond between cysteine residues. Thrombin peptide derivative dimers are typically essentially free of the corresponding monomer, e.g., greater than 95% free by weight and preferably greater than 99% free by weight. Preferably the polypeptides are the same and covalently linked through a disulfide bond.

The thrombin peptide derivative dimers of the present invention include the thrombin peptide derivatives described above. Specifically, thrombin peptide derivatives have less than about fifty amino acids, preferably less than about thirty-three amino acids. Thrombin peptide derivatives also have sufficient homology to the fragment of human thrombin corresponding to thrombin amino acid residues 508-530: AGYKPDEGKRGDACEGDSGGPFV (SEQ ID NO:1) so that the polypeptide activates NPAR.

In a specific embodiment, each thrombin peptide derivative comprising a dimer comprises a polypeptide having the amino acid sequence RGDACX1GDSGGPX2V (SEQ ID NO: 4), or a C-terminal truncated fragment thereof comprising at least six amino acids. More specifically, each thrombin peptide derivative comprises the amino acid sequence of AGYKPDEGKDGDACEGDSGGPFV (SEQ ID NO:5), or a fragment thereof comprising amino acids 10-18. Even more specifically, the thrombin peptide derivative comprises the amino acid sequence AGYKPDEGKRGDACX1GDASGGPX2V (SEQ ID NO:8), or a fragment thereof comprising amino acids 10-18. X1 is Glu or Gln and X2 is Phe, Met, Leu, His or Val. Preferably X1 is Glu, and X2 is Phe. One example of a thrombin peptide derivative of this type is a polypeptide comprising the amino acid sequence AGYKPDEGKRGDACEGDSGGPFV (SEQ ID NO: 1). A further example of a thrombin peptide derivative of this type is a polypeptide having the amino acid sequence H-AGYKPDEGKRGDACEGDSGGPFV-NH₂ (SEQ ID NO:1; TP508). Zero, one, two or three amino acids in the thrombin peptide derivative differ from the amino acid at the corresponding position of the sequences. Preferably, the difference is conservative as for conservative substitutions of the thrombin peptide derivatives.

In another specific embodiment, each thrombin peptide derivative comprising a dimer comprises a polypeptide comprising the amino acid sequence AGYKPDEGKRGDACEGDSGGPFVMKSPFNNRWY (SEQ ID NO:20), or a C-terminal truncated fragment thereof having at least twenty-three amino acids. More preferably, each thrombin peptide derivative comprises the amino acid sequence AGYKPDEGKRGDACX1GDSGGPX2VMKSPFNNRWY (SEQ ID NO:21), or a C-terminal truncated fragment thereof comprising at least twenty-three amino acids. X1 is Glu or Gln and X2 is Phe, Met, Leu, His or Val. Preferably X1 is Glu, and X2 is Phe. One example of a thrombin peptide derivative of this type is a polypeptide comprising the amino acid sequence AGYKPDEGKRGDACEGDSGGPFVMKSPFNNRWY (SEQ ID NO:22). A further example of a thrombin peptide derivative of this type is a polypeptide comprising the amino acid sequence H-AGYKPDEGKRGDACEGDSGGPFVMKSPFNNRWY-NH₂ (SEQ ID NO:23). Zero, one, two or three amino acids in the thrombin peptide derivative differ from the amino acid at the corresponding position of the sequences. Preferably, the difference is conservative as defined for conservative substitutions of the thrombin peptide derivatives.

As used herein an “effective amount” is the quantity of the thrombin peptide derivative described herein that results in an improved clinical outcome of the condition being treated with the thrombin peptide derivative compared with the absence of treatment. The amount of the thrombin peptide derivative administered will depend on the degree, severity, and type of the disease or condition, the amount of therapy desired, and the release characteristics of the pharmaceutical formulation. It will also depend on the subject's health, size, weight, age, sex and tolerance to drugs.

Typically, the thrombin peptide derivative is administered for a sufficient period of time to achieve the desired therapeutic effect. Typically, from about 1 μg per day to about 1 mg per day of the thrombin peptide derivatives (preferably from about 5 μg per day to about 100 μg per day) is administered to the subject in need of treatment, especially for a local means of administration. The thrombin peptide derivatives can also be administered at a dose of from about 0.1 mg/kg/day to about 15 mg/kg/day, with from about 0.2 mg/kg/day to about 3 mg/kg/day being preferred, especially for systemic means of administration. Typical dosages for the thrombin peptide derivative of the invention are also 5-500 mg/day, preferably 25-250 mg/day, especially for systemic means of administration.

Any suitable dosage of peptide or peptides can be administered using the formulations of the present invention. The dosage administered will, of course, vary depending upon known factors, such as the pharmacodynamic characteristics of the peptide, salt, or combination thereof; the age, health, or weight of the subject; the nature and extent of symptoms; the metabolic characteristics of the therapeutic agent and patient, the kind of concurrent treatment; the frequency of treatment; or the effect desired. Generally, the peptide (or, wherein the stable formulation comprises two or more peptides, each of the peptides) is present in the formulation in an amount ranging from about 0.5 mg/mL to about 100 mg/mL (e.g., about 0.5, 1, 2, 3, 4, 5, 6, 7, 8, 9, 10, 15, 20, 25, 30, 35, 40, 45, 50, 55, 60, 65, 70, 75, 80, 85, 90, 95, or 100 mg/mL).

In some embodiments, the peptide is present in the formulation in an amount ranging from about 0.5 mg/mL to about 150 mg/mL. In some embodiments, the peptide is present in the formulation in an amount ranging from about 10 mg/mL to about 125 mg/mL. In other embodiments, the peptide is present in the formulation in an amount ranging from about 20 mg/mL to about 100 mg/mL. In still other embodiments, the peptide is present in said formulation in an amount ranging from about 5 mg/mL to about 150 mg/mL. In yet other embodiments, the peptide is present in the formulation in an amount ranging from about 0.5 mg/mL to about 50 mg/mL. Again, it will be readily apparent to those of skill that the peptide dosage can be varied depending on the peptide used and the disease, disorder or condition to be treated.

### D. Stabilizing Excipients

In certain embodiments, the formulations described herein may be further stabilized to ensure the stability of the peptide incorporated therein. In some embodiments, the stability of the formulation is enhanced by the inclusion of one or more stabilizing agents or stabilizing excipients into the formulation.

In some embodiments, the stabilizing excipient is a cryoprotectant. As shown below in the Examples section, the addition of a cryoprotectant, such as trehalose, protects the peptide formulations of the present invention against instability associated with freeze-thaw cycles. The addition of the cryoprotectant trehalose can promote enhanced thawing of a frozen peptide formulation. This property of enhanced thawing is surprisingly advantageous, particularly in emergency medical situations wherein a peptide formulation of the present invention is frozen and needs to be administered quickly. Thus, in another aspect of the present invention, the stable formulation has an improved freeze-thaw stability, an enhanced thawing rate, and/or an enhanced thawing profile.

In some embodiments, the stabilizing excipient is selected from sugars, starches, sugar alcohols, and mixtures thereof. Examples of suitable sugars for stabilizing excipients include, but are not limited to, trehalose, glucose, sucrose, etc. Examples of suitable starches for stabilizing excipients include, but are not limited to, hydroxyethyl starch (HES). Examples of suitable sugar alcohols for stabilizing excipients include, but are not limited to, mannitol and sorbitol. In some embodiments, the at least one stabilizing excipient (e.g., a sugar, a starch, a sugar alcohol, or a mixture thereof) is capable of enhancing the stability of the peptide during a freeze-thawing process, enhancing the thawing rate of the formulation, or enhancing the thawing profile of the formulation.

In some embodiments, the stabilizing excipient is present in the formulation in an amount ranging from about 1% (w/v) to about 60% (w/v), from about 1% (w/v) to about 50% (w/v), from about 1% (w/v) to about 40% (w/v), from about 1% (w/v) to about 30% (w/v), from about 1% (w/v) to about 20% (w/v), from about 5% (w/v) to about 60% (w/v), from about 5% (w/v) to about 50% (w/v), from about 5% (w/v) to about 40% (w/v), from about 5% (w/v) to about 30% (w/v), from about 5% (w/v) to about 20% (w/v), from about 10% (w/v) to about 60% (w/v), from about 10% (w/v) to about 50% (w/v), from about 10% (w/v) to about 40% (w/v), from about 10% (w/v) to about 30% (w/v), or from about 10% (w/v) to about 20% (w/v). In some embodiments, the stabilizing excipient is present in the formulation in an amount that is about 1%, about 5%, about 10%, about 15%, about 20%, about 25%, about 30%, about 35%, about 40%, about 45%, about 50%, about 55%, or about 60% (w/v).

In some embodiments, the formulation further comprises additional stabilizing agents including, for example, antioxidants, chelators and preservatives. Examples of suitable antioxidants include, but are not limited to, ascorbic acid, cysteine, methionine, monothioglycerol, sodium thiosulphate, sulfites, BHT, BHA, ascorbyl palmitate, propyl gallate, N-acetyl-L-cysteine (NAC), and Vitamin E. Examples of suitable chelators include, but are not limited to, EDTA, tartaric acid and salts thereof, glycerin, and citric acid and salts thereof. Examples of suitable preservatives include, but are not limited to, benzyl alcohols, methyl parabens, propyl parabens, and mixtures thereof.

In some embodiments, the formulation further comprises a stabilizing polyol. Such formulations and materials are described, for example, in U.S. Pat. Nos. 6,290,991 and 6,331,310, the contents of each of which are incorporated by reference herein.

### E. TP508 Formulations

In certain embodiments, the present invention provides a TP508 formulation, the TP508 formulation comprising: a TP508 peptide or salt thereof complexed with a metal ion (for example Zinc (II) and Calcium (II)) and an aqueous solvent (usually Sterile Water for Injection USP or Sterile 0.9% Saline Solution USP, depending on the intended administration route). In some embodiments, the TP508 peptide is present in the formulation in an amount ranging from about 0.5 mg/mL to about 150 mg/mL, or from about 1 mg/mL to about 125 mg/mL. In some embodiments, the formulation further comprises a stabilizing excipient selected from sugars (e.g., trehalose), starches (e.g., hydroxyethyl starch (HES)), and mixtures thereof. The stabilizing excipient may be present in the formulation in an amount ranging from about 1% (w/v) to about 60% (w/v). In some embodiments, the formulation further comprises a co-solvent that depresses the freezing point of the formulation, wherein the co-solvent is selected from ethanol, propylene glycol, glycerol, and mixtures thereof. The co-solvent may be present in the formulation in an amount ranging from about 10% (w/v) to about 50% (w/v).

In other embodiments, the present invention provides a stable TP508 peptide formulation, the TP508 peptide formulation comprising: a TP508 peptide or salt thereof (or TP508 peptide derivative); and an aqueous solvent.

In other particular embodiments, the stable TP508 peptide formulation further comprises a stabilizing excipient that is a sugar, a starch, or a sugar alcohol. For instance, in some embodiments, the TP508 peptide formulation further comprises a glycine buffer and mannitol, or a citrate buffer and mannitol, or a phosphate buffer and mannitol. In some embodiments, the TP508 peptide formulation further comprises a glycine buffer and trehalose, or a citrate buffer and trehalose, or a phosphate buffer and trehalose.

In additional embodiments, the invention includes formulations of peptides conjugated to at least one heterologous moiety, e.g., as described in PCT/US2017/053597, incorporated here by reference. For example, the peptide can be lipidated (e.g., myritoylated, palmitoylated, linked to a C7-C20 lipid moiety), glycosylated, amidated, carboxylated, phosphorylated, esterified, acylated, acetylated, cyclized, pegylated, dimerized, polymerized, attached to a targeting moiety, or otherwise conjugated.

In certain embodiments, the invention relates to a peptide formulation that is stable for a minimum of three days at room temperature. In exemplary embodiments, the invention relates to a formulation that is a clear liquid or gel free for a minimum of three days at room temperature.

In certain embodiments, the invention relates to a peptide formulation that one can administer to a patient through a standard gauge needle. For example, a standard gauge needle is about 27 gauge.

In certain embodiments, the invention relates to a peptide formulation that can be lyophilized to form a solid formulation for reconstitution prior to administration.

Lyophilized forms of formulations described herein are embodiments of the invention. Lyophilized forms are stable at low temperatures, e.g., −70° C. and above, as well as room temperature. Stability of lyophilized formulations has been established at room temperature for longer periods than achievable with the same formulation in solution.

In exemplary embodiments, the invention relates to a formulation that comprises ZnCl₂ as a stabilizer. In certain aspects, ZnCl₂ is present in an aqueous form of the pharmaceutical formulation in a concentration of between about 5 and about 100 mM, preferably between about 5 mM and about 15 mM, and even more preferably about 7 mM to 15 mM based on safety considerations.

In certain aspects, the invention relates to a formulation selected from a formulation comprising a peptide concentration of about 150 mg/mL and an ZnCl₂ concentration of about 5 mM to 15 mM.

In certain embodiments, said aqueous form of the pharmaceutical formulation has a pH of between 4 and 7, preferably between 5 and 7, more preferably about 7.

As used herein, the term “high temperature” shall refer to a storage temperature which is higher than 0° C. Preferably, said high temperature is higher than a temperature selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40° C.

As used herein, the term “long term storage” shall refer to storage of a composition comprising the pharmaceutical formulation for 1 day or more, preferably for 2, 3, 4, 5, 6, 7, 8, 9, 10, 11, 12, 13, 14, 15, 16, 17, 18, 19, 20, 21, 22, 23 days or more, or even 1 month or more.

In certain embodiments, said aqueous form of the pharmaceutical formulation is a solution stable at conditions designed for practical use (i.e., at a temperature suitable for administration) for more than 1 day.

In certain embodiments, the peptide is formulated with an excipient to provide a pharmaceutical composition which composition can be used to treat a disease in a patient or another medical condition.

As used herein, the term “excipient” is intended to mean a therapeutically inactive substance. Excipients can be included in a formulation for a wide variety of purposes including, for example, as a diluent, vehicle, buffer, stabilizer, tonicity agent, bulking agent, surfactant, cryoprotectant, lyoprotectant, anti-oxidant, metal ion source, chelating agent and/or preservative. Excipients include, for example, polyols such as sorbitol or mannitol; sugars such as sucrose, lactose or dextrose; polymers such as polyethylene glycol; salts such as NaCl, KCl or calcium phosphate, amino acids, for example, proline, glycine or methionine, surfactants, metal ions, buffer salts such as glutamate, acetate or aspartate, preservatives and polypeptides such as human serum albumin, as well as saline and water. Excipients can comprise sugars, for example sugar alcohols, reducing sugars, non-reducing sugars and sugar acids. Excipients are well known in the art and can be found described in, for example, Wang W., Int. J. Pharm. 185:129-88 (1999) and Wang W., Int. J. Pharm. 203:1-60 (2000).

In various embodiments, the formulation can include one or more excipients. One potential role of an included excipient is to provide stabilization of the biopharmaceutical against stresses that can occur during manufacturing, shipping and storage. To accomplish this, at least one excipient can function as a buffer, stabilizer, tonicity agent, bulking agent, surfactant, cryoprotectant, lyoprotectant, antioxidant, metal ion source, chelating agent and/or preservative. In addition, at least one excipient can function as a diluent and/or vehicle or be employed to reduce viscosity in high concentration formulations to enable their delivery and/or enhance patient convenience.

Various excipients that can be useful in either a liquid or lyophilized formulation comprise, fucose, cellobiose, maltotriose, melibiose, octulose, ribose, xylitol, arginine, histidine, glycine, alanine, methionine, glutamic acid, lysine, imidazole, glycylglycine, mannosylglycerate, Triton X-100, Pluronic F-127, cellulose, cyclodextrin, dextran (10, 40 and/or 70 kD), polydextrose, maltodextrin, ficoll, gelatin, hydroxypropylmethyl cellulose, sodium phosphate, potassium phosphate, ZnCl₂, zinc, zinc oxide, sodium citrate, trisodium citrate, tromethamine, copper, fibronectin, heparin, human serum albumin, protamine, glycerin, EDTA, m-cresol, benzyl alcohol and phenol. Various excipients known in the art are described in, for example, Wang W., Int. J. Pharm. 185:129-88 (1999) and Wang W., Int. J. Pharm. 203:1-60 (2000).

As used herein, “preservative” refers to a compound that can be added to a formulation to help maintain stability of the peptide over time by, for example, reducing the impact of bacterial, fungal or other unwanted organic growth. The addition of a preservative may also facilitate the production of a multi-use (multiple-dose) formulation. Examples of potential preservatives include, but are not limited to, octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as, for example, phenol, butyl and benzyl alcohol, alkyl parabens such as methyl or propyl paraben, catechol, resorcinol, cyclohexanol, 3-pentanol, and m-cresol.

High concentration formulations are required for effective use of therapeutic peptides via any parenteral route, for example via subcutaneous injection or intravenous injection for treatment of various disorders. The disclosed formulations are also intended for treatment of systemic disorders, the treatment of which may require repeated administrations via subcutaneous injection or intravenous injection. Without the availability of a high concentration formulation, systemic administration via subcutaneous injection might not be possible or practical.

Stable solutions with low viscosity, which are free of small particles, are less likely to form aggregates. Such solutions are generally considered to be the result of good formulations. Such solution properties are crucial for peptide formulation at higher concentrations (and especially concentrations above 25 mg/mL).

As used herein, “viscosity” refers to “kinematic viscosity” or “absolute viscosity.” “Kinematic viscosity” is a measure of the resistive flow of a fluid under the influence of gravity. Absolute Viscosity=Kinematic Viscosity×Density. Viscosity is concentration dependent. Several previously described formulations consider the highest attainable concentration of peptide to be the concentration at which the viscosity of the solution reaches 20 cP. Using the materials and methods described herein, the viscosity of the formulations described herein reaches 20 cP at a peptide concentration of 10-100 mg/mL. Formulations of therapeutic peptides typically reach a viscosity of 20 cP at concentrations usually less than 100 mg/mL, for example in the range of about 25 to about 75 mg/mL.

Briefly, sugar alcohols, also known as polyols, polyhydric alcohols, or polyalcohols, are hydrogenated forms of carbohydrate having a carbonyl group reduced to a primary or secondary hydroxyl group. Polyols can be used as stabilizing excipients and/or isotonicity agents in both liquid and lyophilized formulations. Polyols can protect biopharmaceuticals from both physical and chemical degradation pathways. Examples of sugar alcohols can include sorbitol, glycerol, mannitol, xylitol, maltitol, lactitol, erythritol and threitol.

Reducing sugars can comprise, for example, sugars with a ketone or aldehyde group and contain a reactive hemiacetal group, which allows the sugar to act as a reducing agent. Specific examples of reducing sugars include fructose, glucose, glyceraldehyde, lactose, arabinose, mannose, xylose, ribose, rhamnose, galactose and maltose. Non-reducing sugars can comprise an anomeric carbon that is an acetal and is not substantially reactive with amino acids or polypeptides to initiate a Maillard reaction. Specific examples of non-reducing sugars include sucrose, trehalose, sorbose, sucralose, melezitose and raffinose. Sugar acids include, for example, saccharic acids, gluconate and other polyhydroxy sugars and salts thereof.

Buffers or buffers in combination with excipients can maintain the pH of liquid formulations throughout product shelf-life and maintain the pH of lyophilized formulations during the lyophilization process and upon reconstitution.

Tonicity agents and/or stabilizers included in liquid formulations can be used, for example, to provide isotonicity, hypotonicity or hypertonicity to a formulation such that it is suitable for administration. Such excipients also can be used to facilitate maintenance of a peptide's structure and/or to minimize electrostatic, solution protein-protein interactions. Examples of tonicity agents and/or stabilizers can include polyols, salts and/or amino acids.

Anti-oxidants are useful in liquid formulations to control protein oxidation and also can be used in lyophilized formulations to retard oxidation reactions.

Metal ions can be included in a liquid formulation, for example, as a co-factor and divalent cations such as zinc and magnesium can be utilized in suspension formulations. Chelating agents included in liquid formulations can be used, for example, to inhibit metal ion catalyzed reactions. With respect to lyophilized formulations, metal ions also can be included, for example, as a co-factor. Although chelating agents are generally omitted from lyophilized formulations, they also can be included as desired to reduce catalytic reactions during the lyophilization process and upon reconstitution.

Stability of a formulation refers to the retention of structure and/or function and/or biological activity of a biopharmaceutical within the formulation. The retention of structure and/or function and/or biological activity does not need to be 100%. Measurement of the stability of a formulation can be a comparative measure. Therefore, if one formulation is said to be more stable or have greater stability than another, the formulation with greater stability has retained a greater percentage of a desired characteristic being investigated than the other formulation, unless the characteristic being considered is a negative characteristic. If the characteristic is a negative characteristic, then the formulation with greater stability will have less of that characteristic. For example, formulation A is more stable than formulation B if it has a lower propensity to form a gel than formulation B during storage.

In various embodiments, the stability of a peptide within a formulation can comprise the retention of physical and/or chemical stability. Biopharmaceutical stability can be assessed by, for example, determining whether the biopharmaceutical has been subjected to a physical degradation and/or chemical degradation pathway, including chemical modification of its structure. Retention of stability can also be measured, for example, in terms of the extent of gelling, after storage at different temperatures or after multiple freeze-thaw cycles. These measurements can reflect the amount of peptide aggregation.

Stability of a peptide formulation can be evaluated by multiple criteria as described herein. In some variations, stability is evinced by purity. One indicia of purity is quantification of major and minor forms, e.g., with chromatographic analysis (such as RP-HPLC or ion exchange HPLC). In some variations, purity is at least 90%, 91%, 92%, 93%, 94%, 95%, 96%, 97% 98%, or 99% as evaluated by the areas of major versus minor chromatographic peaks. Purities in the range of 90-99.5%, including integer and half-integer subranges thereof, are specifically contemplated. In some variations, stability is evinced by properties of the peptide in solution, including stable pH, minimal or absence of turbidity (e.g., when evaluated at 400-700 nm), minimal or absence of particles, and/or minimal or absence of discoloration. In some variations, stability is evinced by retention of biological activity, e.g., in cell based assays or animal models. In some variations, purity is evinced by a plurality of these criteria.

Preservatives in liquid formulations can be used, for example, to protect against microbial growth and are particularly beneficial in multi-dose formulations. In lyophilized formulations, preservatives are generally included in the reconstitution diluent. Benzyl alcohol is a specific example of a preservative useful in a formulation of the invention.

As used herein, the term “surfactant” is intended to mean a substance that functions to reduce the surface tension of a liquid in which it is dissolved. Surfactants can be included in a formulation for a variety of purposes including, for example, to prevent or control aggregation, particle formation and/or surface adsorption in liquid formulations or to prevent or control these phenomena during the lyophilization and/or reconstitution process in lyophilized formulations. Surfactants include, for example, amphipathic organic compounds that exhibit partial solubility in both organic solvents and aqueous solutions. General characteristics of surfactants include their ability to reduce the surface tension of water, reduce the interfacial tension between oil and water and also form micelles. Surfactants of can include non-ionic and ionic surfactants. Surfactants are known in the art and can be found described in, for example, Randolph T. W. and Jones L. S., Surfactant-protein interactions. Pharm Biotechnol. 13:159-75 (2002).

Non-ionic surfactants can include, for example, alkyl poly(ethylene oxide), alkyl polyglucosides such as octyl glucoside and decyl maltoside, fatty alcohols such as cetyl alcohol and oleyl alcohol, cocamide MEA, cocamide DEA, and cocamide TEA. Specific examples of non-ionic surfactants include the polysorbates including, for example, polysorbate 20, polysorbate 28, polysorbate 40, polysorbate 60, polysorbate 65, polysorbate 80, polysorbate 81, polysorbate 85 and the like; the poloxamers including, for example, poloxamer 188, also known as poloxalkol or poly(ethylene oxide)-poly(propylene oxide), poloxamer 407 or polyethylene-polypropylene glycol and the like, and polyethylene glycol (PEG). Polysorbate 20 is synonymous with TWEEN 20, sorbitan monolaurate and polyoxyethylenesorbitan monolaurate.

Ionic surfactants can include, for example, anionic, cationic and zwitterionic surfactants. Anionic surfactants include, for example, sulfonate-based or carboxylate-based surfactants such as soaps, fatty acid salts, sodium dodecyl sulfate (SDS), ammonium lauryl sulfate and other alkyl sulfate salts. Cationic surfactants include, for example, quaternary ammonium-based surfactants such as cetyl t trimethylammonium bromide (CTAB), other alkyltrimethylammonium salts, cetyl pyridinium chloride, polyethoxylated tallow amine (POEA) and benzalkonium chloride. Zwitterionic or amphoteric surfactants include, for example, dodecyl betaine, dodecyl dimethylamine oxide, cocamidopropyl betaine and coco ampho carboxy glycinate.

In various embodiments, the formulation forms a sustained release matrix. In various embodiments, the formulation forms a sustained-release matrix upon subcutaneous injection or intramuscular injection. In various embodiments, the release of said drug substance is over a period of greater than about four hours. In various embodiments, the release of said drug substance is over a period of greater than about eight hours. In various embodiments, the release of said drug substance is over a period of greater than about twelve hours. In various embodiments, the release of said drug substance is over a period of greater than about twenty-four hours. In various embodiments the release of said drug substance is longer than a period of seven 7 days. In various embodiments, the pharmacological effect from at least one of said drug substance lasts at least about four hours. In various embodiments, the pharmacological effect from at least one of said drug substance lasts at least about eight hours. In various embodiments, the pharmacological effect from at least one of said drug substance lasts at least about twelve hours. In various embodiments, the pharmacological effect from at least one of said drug substance lasts at least about twenty-four hours.

“Sustained release dosage forms” or “sustained release matrix” mean forms designed to release a drug at a predetermined rate by maintaining a constant drug level for a specific period of time with minimum side effects. The basic rationale of sustained release drug delivery system optimizes the biopharmaceutical, pharmacokinetic and pharmacodynamics properties of a drug in such a way that its utility is maximized, side-effects are reduced and cure and/or treatment of the disease is better achieved.

Determination of an effective amount or dose is well within the capability of those skilled in the art, especially in light of the detailed disclosure provided herein. Generally, the formulations to deliver these doses may contain one, two, three, four, or more peptides, or peptide analogs (collectively “peptide,” unless peptide analogs are expressly excluded), wherein each peptide is present at a concentration from about 0.1 mg/mL up to the solubility limit of the peptide in the formulation. This concentration is preferably from about 1 mg/mL to about 150 mg/mL. In certain aspects, the concentration is about 1 mg/mL, about 2 mg/mL, about 2.5 mg/mL, about 3 mg/mL, about 4 mg/mL, about 5 mg/mL, about 7.5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 100 mg/mL, about 110 mg/mL, about 120 mg/mL, about 130 mg/mL, about 140 mg/mL, or about 150 mg/mL.

The formulations of the present invention may be used for various administration routes, including, but not limited to, subcutaneous, intradermal, intramuscular, intraocular, intranasal, buccal, transdermal or intravenous administration (e.g., by injection or by infusion). In some embodiments, the formulation is administered subcutaneously. The formulations can also be delivered transdermally, such as by topically applying the composition to healthy or wounded skin (e.g., spreading the composition on skin or loading the composition onto a dermal patch and attaching the dermal patch to the skin).

The formulations of the present disclosure can be administered by infusion or by injection using any suitable device. For example, a formulation of the present invention may be placed into a syringe (e.g., a pre-filled syringe), a pen injection device, an auto-injector device, or a pump device. In some embodiments, the injection device is a multi-dose injector pump device or a multi-dose auto-injector device. The formulation is presented in the device in such a fashion that the formulation is readily able to flow out of the needle upon actuation of an injection device, such as an auto-injector, in order to deliver the peptide drugs. Suitable pen/auto injector devices include, but are not limited to, those pen/auto injection devices manufactured by Becton-Dickenson, Swedish Healthcare Limited (SHL Group), YpsoMed Ag, and the like. Suitable pump devices include, but are not limited to, those pump devices manufactured by Tandem Diabetes Care, Inc., Delsys Pharmaceuticals and the like.

In some embodiments, the formulations of the present invention are provided ready for administration in a vial, a cartridge, or a pre-filled syringe.

### II. EXAMPLES

The following examples are included to represent certain aspects of the invention. It should be appreciated by those of skill in the art that the techniques disclosed in the examples represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### TP508 Formulation Studies

Unless otherwise indicated, the studied formulations were prepared at 150 mg/mL (corresponding to 65 mM) of peptide along with the stated amounts of excipients/metal salts (ions) in Sterile Water for Injection or the equivalent. With the exception of short term stability studies performed at 5° C. and 25° C./60% RH (Table 2, entries 61-64), all formulation studies described in the following tables were performed using samples stored at 40°±2° C./75±5% RH (hereafter referred to simply as 40° C.) for seven (7) days followed by analysis. The use of the 40° C. condition allowed for observation of formulation degradation in a practical timeframe. The purity determinations in Table 2 were performed by reversed phase high performance liquid chromatography (RP-HPLC) according to client method TM-DP-0012; a fresh control containing 150 mg/mL TP508 in water was run concurrently and the peptide peak areas of the samples were ratioed against the standard purity to provide the formulation purity in wt % of TP508. From previous studies, it has been established that the only significant impurity is usually the dimer resulting from cysteine-cysteine oxidative coupling, so a decrease in peptide purity generally corresponds to a similar increase in dimer and vice-versa.
Table 1 provides preliminary characteristics for initial formulations containing 150 mg/mL peptide, stored at 40° C. for 7 days and analyzed for appearance (T0), osmolality (T0) and DLS/SLS (T1 week), the latter two mainly for general informational purposes. These are briefly discussed below.
Appearance. All entries in Table 1 consisted of clear, colorless solutions with no visible particles.
Osmolality. As expected, the entries in Table 1 were mostly hyperosmotic, consistent with the number of solute particles. This has not been analyzed in subsequent studies.
DLS/SLS. The tendency towards particle aggregation was assessed by Dynamic/Static Light Scattering (DLS/SLS) using the Stokes Einstein equation to calculate the hydrodynamic radius of the particle(s) in solution. PDI values ≤0.25 correspond to monodisperse sample and PDI values above 0.25 correspond to polydisperse samples. The results in Table 1 suggest that aggregation is not occurring. Additionally, no significant changes in fluorescence emission upon heating were observed. The derivate trace of the Barycentric Mean (BCM) against the temperature can be used to identify the melting temperature or Tₘ of proteins. However, this calculation requires a clear inflection point in the derivative plot of the BCM which was not present for the TP508 preparations. Additionally, there are no significant changes in SLS counts at 266 nm and 473 nm upon heating. The temperature of aggregation, which is the temperature at 10% of the apex for the 1^{st} derivative of the SLS plot, requires a clear inflection point which was not present for the TP508 preparations. Therefore, Tₘ and T_{agg} were not calculated, and DLS/SLS will not be performed in subsequent studies.

TABLE 1
Preliminary Characteristics of Selected Excipients and Metal Salts on TP508 Stability

| | | | | | | After 40° C./75% RH for 7 Days | | | |
|---|---|---|---|---|---|---|---|---|---|
| | | | | At t₀ | | Z-Average | | Polydispersity | |
| | [TP508] | | Metal | | Osmolality | Diameter (nm) | | Index (PDI) | |
| Entry | (mg/mL) | Excipients | Salts | Appearance | (mOsm/kg) | 20° C. | 95° C. | 20° C. | 95° C. |
| 1 | 150 | 0.9% NaCl | None | A | 517 | 6.9 | 3.9 | 0.04 | 0.07 |
| 2 | 150 | DPBS | None | A | 524 | 7.0 | 4.6 | 0.04 | 0.05 |
| 3 | 150 | PBS | None | A | 524 | 7.0 | 4.5 | 0.03 | 0.06 |
| 4 | 150 | 200 mM | None | A | 410 | 6.3 | 4.6 | 0.08 | 0.11 |
| | | Histidine | | | | | | | |
| 5 | 150 | 100 mM | None | A | 325 | 6.8 | 4.7 | 0.06 | 0.08 |
| | | Histidine | | | | | | | |
| 6 | 150 | 50 mM | None | A | 282 | 6.1 | 4.3 | 0.07 | 0.08 |
| | | Histidine | | | | | | | |
| 7 | 150 | 50 mM | None | A | 531 | 6.4 | 4.5 | 0.07 | 0.07 |
| | | Histidine, 130 | | | | | | | |
| | | mM Arginine | | | | | | | |
| 8 | 150 | 50 mM | None | A | 351 | 6.1 | 4.2 | 0.07 | 0.08 |
| | | Histidine, 65 | | | | | | | |
| | | mM Methionine | | | | | | | |
| 9 | 150 | 50 mM | 65 mM | A | 437 | 6.3 | 3.9 | 0.06 | 0.08 |
| | | Histidine | MgCl₂ | | | | | | |
| 10 | 150 | 50 mM | 65 mM | A | 387 | 4.8 | 2.9 | 0.09 | 0.09 |
| | | Histidine | ZnCl₂ | | | | | | |
| 11 | 150 | 50 nM Histidine, | None | A | 288 | 6.0 | 4.3 | 0.06 | 0.08 |
| | | 10 μM EDTA | | | | | | | |
| 12 | 150 | 50 nM Histidine, | None | A | 340 | 5.3 | 3.7 | 0.09 | 0.10 |
| | | 65 mM Cysteine | | | | | | | |

A: Clear, colorless solution, no visible particles

Table 2 provides a more extensive survey of various excipients (metal salts, buffers, antioxidants), pH, ionic strengths, peptide concentration and short term stability studies at 5° C. and 25° C./60% RH towards optimizing peptide purity, with the main points discussed below.
Metal Salts. The results in Table 2 showed suboptimal purity of TP508 after storage at 40° C. for one week in the different formulations except for those containing zinc chloride, which showed good stability of TP508 in the presence and absence of histidine and citrate buffer (entries 10, 13-20, 29, 33, 46, 52, 53, 56). Calcium chloride alone was not enough to improve the stability of the peptide after storage at 40° C. for one week but gave better results in combination with citrate and EDTA (Entries 23-28, 30, 35, 36). Magnesium chloride (Entry 9) did not stabilize the peptide. All formulations containing Fe(II) or Fe(III) (Entries 38-45) did not stabilize the peptide with formation of brown liquid, usually too dark to assess clarity or particulates.
Buffers. Two buffers (histidine, citrate) were mainly used in the Table 2 formulations, with arginine, EDTA and cysteine less commonly used as additives. The effect of these buffers is unclear, although the use of histidine with zinc chloride formulations appears to give improved peptide purity versus citrate.
Antioxidants. To assess the effects of antioxidants on peptide monomeric purity, two antioxidants were assessed, one formulation with 65 mM methionine and the second formulation with 5 vol % DMSO (Entries 54-55). These formulations did not enhance peptide purity and, for the DMSO entry, resulted in total degradation of the peptide.
pH. The results of pH feasibility testing performed initially to determine the buffer strength needed to maintain the desired pH had shown that due to the buffer capacity of the peptide (aqueous pH about 4), the desired pH would be challenging to obtain at the 150 mg/mL target peptide concentration. Therefore, the preformulation screening focused initially on evaluation/screening of various additives on TP508 purity without pH adjustment. To assess the effects of pH on peptide purity, TP508 was prepared at a concentration of 150 mg/mL in 50 mM histidine with 65 mM zinc chloride was intentionally adjusted to the following nominal pH values: 2.0, 3.0, 6.0, 7.0, and 8.0 (Entries 47-51) (pH 4-5 were not considered based on preclinical animal studies). The results in the pH range of 6-8 showed nearly 100% peptide purity relative to the control, demonstrating a significant relationship between pH and peptide purity for formulations containing zinc chloride. This is addressed further in Tables 3-7.
Ionic Strength. To assess the effects of ionic strength on peptide monomeric purity, the divalent metal ion concentrations (mainly zinc and calcium) were varied between 4 mM and 260 mM (numerous entries in Table 2). The sample purity was sometimes affected in an unpredictable manner due to contributions from added excipients. Additionally, monovalent salts (sodium chloride or potassium chloride) were added to the formulations at the same molarity of the peptide (Entries 52-53). The results did not show any enhancement of peptide purity versus the formulation without the added monovalent salts (Entry 10).
Peptide Concentration. The objective was to assess the monomeric stability of the peptide in solution when the concentration is reduced. For this purpose, 50 mg/mL TP508 was prepared in 50 mM histidine with 65 mM zinc chloride. Following the initial dilution, a secondary dilution was prepared to 10 mg/mL TP508 (Entries 57-58). The 50 mg/mL peptide concentration provided results similar to those at the 150 mg/mL peptide concentration, while the 10 mg/mL peptide concentration resulted in no observed peptide remaining.
Stability Study. A short term stability study was performed for 150 mg/mL TP508 in 50 mM histidine with 65 mM zinc chloride stored at both 5° C. and 25° C. for 2 weeks and 1 month (Entries 61-64). The results showed that the product remained stable throughout the studies.

TABLE 2
Evaluation/Screening of Various Excipients and Metal Ions on TP508 Stability

| | | | | After 40° C./75% RH for 7 Days | | |
|---|---|---|---|---|---|---|
| | [TP508] | | | | Purity | |
| Entry | (mg/mL) | Excipients | Metal Salts | Appearance | (Wt %) | pH |
| 1 | 150 | 0.9% NaCl | None | A | 1.3 | 4.2 |
| 2 | 150 | DPBS | None | A | 1.5 | 4.3 |
| 3 | 150 | PBS | None | A | 1.4 | 4.3 |
| 4 | 150 | 200 mM Histidine | None | A | 4.1 | 5.2 |
| 5 | 150 | 100 mM Histidine | None | A | 2.9 | 4.7 |
| 6 | 150 | 50 mM Histidine | None | A | 6.8 | 4.5 |
| 7 | 150 | 50 mM Histidine, 130 mM | None | A | 1.7 | 5.2 |
| | | Arginine | | | | |
| 8 | 150 | 50 mM Histidine, 65 mM | None | A | 9.6 | 4.5 |
| | | Methionine | | | | |
| 9 | 150 | 50 mM Histidine | 65 mM MgCl₂ | A | 4.7 | 4.4 |
| 10 | 150 | 50 mM Histidine | 65 mM ZnCl₂ | A | 68.7 | 4.2 |
| 11 | 150 | 50 mM Histidine, 10 μM | None | A | 11.3 | 4.5 |
| | | EDTA | | | | |
| 12 | 150 | 50 mM Histidine, 65 mM | None | A | 10.9 | 4.5 |
| | | Cysteine | | | | |
| 13 | 150 | None | 65 mM ZnCl₂ | A | 67.4 | 4.1 |
| 14 | 150 | 10 mM Histidine | 65 mM ZnCl₂ | A | 68.1 | 4.1 |
| 15 | 150 | 30 mM Histidine | 65 mM ZnCl₂ | A | 69.4 | 4.2 |
| 16 | 150 | 50 mM Histidine | 16.3 mM | B | 57.4 | 4.4 |
| | | | ZnCl₂ | | | |
| 17 | 150 | 50 mM Histidine | 32.5 mM | A | 68.2 | 4.3 |
| | | | ZnCl₂ | | | |
| 18 | 150 | 50 mM Histidine | 65 mM ZnCl₂ | A | 70.1 | 4.2 |
| 19 | 150 | 50 mM Histidine | 130 mM | A | 69.7 | 4.1 |
| | | | ZnCl₂ | | | |
| 20 | 150 | 50 mM Histidine | 260 mM | A | 67.7 | 3.9 |
| | | | ZnCl₂ | | | |
| 21 | 150 | None | 65 mM CaCl₂ | C | 3.1 | 4.3 |
| 22 | 150 | 50 mM Histidine | 65 mM CaCl₂ | B | 7.5 | 4.4 |
| 23 | 150 | 50 mM Citrate | 16.3 mM | A | 20.5 | 4.0 |
| | | | CaCl₂ | | | |
| 24 | 150 | 50 mM Citrate | 32.5 mM | A | 23.9 | 3.9 |
| | | | CaCl₂ | | | |
| 25 | 150 | 50 mM Citrate | 65 mM CaCl₂ | A | 31.4 | 3.8 |
| 26 | 150 | 50 mM Citrate | 130 mM | E | 39.4 | 3.5 |
| | | | CaCl₂ | | | |
| 27 | 150 | 50 mM Citrate | 260 mM | F | 22.3 | 3.4 |
| | | | CaCl₂ | | | |
| 28 | 150 | 50 mM Citrate | 65 mM ZnCl₂ | A | 39.5 | 3.6 |
| 29 | 150 | 50 mM Histidine + 10 μM | 260 mM | A | 67.3 | 3.9 |
| | | EDTA | ZnCl₂ | | | |
| 30 | 150 | 50 mM Citrate + 10 μM | 260 mM | E | 58.1 | 3.4 |
| | | EDTA | CaCl₂ | | | |
| 31 | 150 | 50 mM Histidine + 10 μM | None | B | 33.7 | 4.5 |
| | | EDTA + 65 mM Cysteine | | | | |
| | | (Table 8 ends) | | | | |
| 32 | 150 | 50 mM Histidine + 20 μM | None | C | 15.2 | 4.5 |
| | | EDTA | | | | |
| 33 | 150 | 50 mM Histidine + 20 μM | 260 mM | D | 69.1 | 3.9 |
| | | EDTA | ZnCl₂ | | | |
| 34 | 150 | 50 mM Histidine + 20 μM | None | F | 20.9 | 4.5 |
| | | EDTA + 65 mM Cysteine | | | | |
| 35 | 150 | 50 mM Citrate + 20 μM | 260 mM | F | 44.7 | 3.5 |
| | | EDTA | CaCl₂ | | | |
| 36 | 150 | 50 mM Citrate + 10 μM | 130 mM | F | 37.4 | 3.6 |
| | | EDTA | CaCl₂ | | | |
| 37 | 150 | 50 mM Citrate + 10 μM | 65 mM ZnCl₂ | | 45.1 | 3.7 |
| | | EDTA | | | | |
| 38 | 150 | 50 mM Histidine | 65 mM FeCl₂ | I | 0.3 | 4.1 |
| 39 | 150 | 50 mM Histidine + 10 μM | 65 mM FeCl₂ | I | 0.3 | 4.1 |
| | | EDTA | | | | |
| 40 | 150 | 50 mM Citrate | 65 mM FeCl₂ | I | 0.3 | 3.2 |
| 41 | 150 | 50 mM Citrate + 10 μM | 65 mM FeCl₂ | I | 0.7 | 3.2 |
| | | EDTA | | | | |
| 42 | 150 | 50 mM Histidine | 65 mM FeCl₃ | I | 0.2 | 3.7 |
| 43 | 150 | 50 mM Histidine + 10 μM | 65 mM FeCl₃ | I | 0.4 | 3.7 |
| | | EDTA | | | | |
| 44 | 150 | 50 mM Citrate | 65 mM FeCl₃ | H | 0.2 | 2.7 |
| 45 | 150 | 50 mM Citrate + 10 μM | 65 mM FeCl₃ | G | N/A | 2.6 |
| | | EDTA | | | | |
| 46 | 150 | None | 65 mM ZnCl₂ | D | 62.8 | 4.1 |
| 47 | 150 | pH 2.0 (target) | 65 mM ZnCl₂ | D | 52.8 | 2.4 |
| 48 | 150 | pH 3.0 (target) | 65 mM ZnCl₂ | D | 45.7 | 2.9 |
| 49 | 150 | pH 6.0 (target) | 65 mM ZnCl₂ | D | 98.0 | 5.6 |
| 50 | 150 | pH 7.0 (target) | 65 mM ZnCl₂ | D | 100.6 | 6.8 |
| 51 | 150 | pH 8.0 (target) | 65 mM ZnCl₂ | E | 97.8 | 7.4 |
| 52 | 150 | None | 65 mM | D | 65.5 | 4.1 |
| | | | ZnCl₂ + 65 | | | |
| | | | mM NaCl | | | |
| 53 | 150 | None | 65 mM ZnCl₂ + | E | 64.4 | 4.1 |
| | | | 65 mM KCl | | | |
| 54 | 150 | 65 mM Methionine | 65 mM ZnCl₂ | D | 55.5 | 4.1 |
| 55 | 150 | 5% DMSO | 65 mM ZnCl₂ | D | 0.0 | 4.2 |
| 56 | 150 | None | 20 mM ZnCl₂ | F | 57.4 | 4.2 |
| 57 | 150 | None | 4 mM ZnCl₂ | F | 0.0 | 4.3 |
| 58 | 150 | None | 65 mM ZnCl₂ | A | 91.1 | 4.0 |
| 59 | 50 | None | 20 mM ZnCl₂ | D | 71.4 | 4.2 |
| 60 | 10 | None | 4 mM ZnCl₂ | D | 0 | 4.5 |
| 61 | 150 | 50 mM Histidine, 5° C., 2 wks | 65 mM ZnCl₂ | D | 94.4 | 4.2 |
| 62 | 150 | 50 mM Histidine, 5° C., 1 mo | 65 mM ZnCl₂ | E | 98.0 | 4.2 |
| 63 | 150 | 50 mM Histidine, 25° C., 2 | 65 mM ZnCl₂ | F | 79.3 | 4.2 |
| | | wks | | | | |
| 64 | 150 | 50 mM Histidine, 25° C., 1 mo | 65 mM ZnCl₂ | F | 76.9 | 4.2 |

A: Clear, colorless solution, no visible particles
B: Clear, yellowish solution, no visible particles
C: Clear, yellowish solution, small particles
D: Clear, colorless solution, small particles
E: Clear or slightly cloudy, colorless solution, large particles or aggregates
F: Clear or slightly cloudy, yellowish solution, large particles or aggregates
G: Clear, light brown liquid, no visible particles
H: Clear, brown liquid, some particles
I: Dark brown liquid, too dark to assess clarity or particles

Tables 3-7. The purpose of these studies was to find (a) the minimum concentration of ZnCl₂ ([ZnCl₂]) resulting in the maximum peptide assay; (b) the formulation pH at which the maximum peptide assay is realized; and (c) whether the zinc chloride concentration and pH are dependent upon each other. The potential interrelation of these attributes is based on the hypothesis that ZnCl₂ stabilizes TP508 through chelation of the peptide which fixes it in a conformation that reduces the probability of oxidative dimerization; the reduced conformational space could also influence the pKa of the peptide itself (pKa of the peptide is around 4.5). It is therefore possible that varying [ZnCl₂] has an influence on the effects of varying the pH. For this reason, a statistical design of experiments (DOE) was employed to address these points. The DOE tool used was a Box-Wilson Central Composite Inscribed (CCI) design (α=(√2)/2) with two factors (concentration of ZnCl₂, pH), two levels (5 mM ZnCl₂, 65 mM ZnCl₂ and pH 6, pH 8) and a center point (35 mM ZnCl₂, pH 7). After normalizing the design space to a scale of [−1;1], a total of 11 samples (three repeats of the center point and 8 samples with systematic variation of [ZnCl₂] and pH) were analyzed after storage at 40° C. for 7 days. As every sample was analyzed in duplicate, this resulted in a total of 22 sample injections; the average values are shown in Table 3, with surface response mapping in Table 5 (monomeric peptide) and Table 6 (dimeric peptide). The 22 sample injection slots were randomized to exclude injection sequence as a confounding factor. Samples for these studies were prepared as previously described, using dilute NaOH or HCl to adjust the pH. The results from these studies are summarized as follows:
   There appears to be interaction between the [ZnCl₂] and pH. The relative area of the monomeric peptide peak is reduced at low ZnCl₂ concentration (5-13.7 mM) and/or high pH.
   Samples with 65 mM ZnCl₂ had larger changes in pH following elevated temperature storage for one week. There were no observed pH changes in other samples.
   The mass balance of monomeric plus dimeric peptide was nearly 100% in all cases (Table 5 and Table 6), indicating that dimerization was the only significant degradation route.
   All subvisible particle size distribution data (≥10 μm, ≥25 μm) meet the USP <788> acceptance criteria. Lower pH appears to be associated with fewer visible particles present. Visible particles are unlikely to be correlated with the precipitation of zinc hydroxide; it is possible that the presence of few visible white fibers is caused by the formation of particles that are intrinsic to the drug substance.
From the data in Table 3, and to minimize the administered dose of zinc chloride for safety considerations, the purpose of this study was to fine-tune the minimum acceptable concentration of ZnCl₂ in the range of 7-15 mM zinc chloride with initial pH 5.0-7.0. The results, performed with the same statistical design as used for Table 3, are shown in Table 4 with surface response mapping in Table 7. From this study, peptide stability was decreased with increasing pH, with this effect most noticeable at 5 mM zinc chloride concentration. At comparable pH and zinc chloride concentrations, the results in Tables 3-4 follow similar trends, with slightly lower overall results in Table 4 presumably due to normal variability upon the accelerated 40° C. storage for one week. Peptide formulations can include stable aqueous compositions comprising 50 to 200 mg/ml TP508 or a TP508 derivative and ZnCl₂ or another suitable zinc salt at a concentration of 7 mM to 260 mM at a pH of 5 to 8.5. The peptide formulations can include excipients such as histidine and citrate. These stabilized formulations provide a solution to the problems associated with therapeutic TP508 formulation stability.

TABLE 3
DOE Study Results ([ZnCl₂] = 5-65 mM, pH = 5.5-8.9)

| | | | | After 7 Days at 40° C. | | | | | | |
|---|---|---|---|---|---|---|---|---|---|---|
| | | | | | | Purity | | | | |
| | [TP508] | [ZnCl₂], | pH | | | (area % vs | | | Minimum | Maximum |
| Entry | (mg/mL) | mM | (t₀) | pH | Appearance | control) | ≥10 μm | ≥25 μm | size (μm) | size (μm) |
| 65 | 150 | 35 | 7.0 | 6.4 | B | 94.3 | 1956 | 278 | 1.1 | 121 |
| 66 | 150 | 35 | 7.0 | 7.0 | B | | 1386 | 184 | 1.1 | 105 |
| 67 | 150 | 35 | 7.0 | 6.6 | C | | 1262 | 266 | 1.1 | 107 |
| 68 | 150 | 65 | 8.5 | 5.9 | D | 91.6 | 2451 | 529 | 1.1 | 244 |
| 69 | 150 | 65 | 5.5 | 7.5 | A | 90.7 | 1490 | 312 | 1.1 | 225 |
| 70 | 150 | 5 | 8.5 | 8.9 | E | 54.7 | 673 | 95 | 1.1 | 92 |
| 71 | 150 | 5 | 5.5 | 5.5 | A | 3.2 | 698 | 145 | 1.1 | 125 |
| 72 | 150 | 56.2 | 8.1 | 7.6 | A | 93.0 | 932 | 236 | 1.1 | 230 |
| 73 | 150 | 56.2 | 5.9 | 5.5 | C | 92.4 | 1517 | 159 | 1.1 | 115 |
| 74 | 150 | 13.7 | 8.1 | 7.9 | A | 64.2 | 1553 | 207 | 1.1 | 228 |
| 75 | 150 | 13.7 | 5.9 | 5.7 | A | 93.4 | 975 | 134 | 1.1 | 106 |

A: no particles present
B: few, short, white fibers present
C: few, short, translucent fibers present
D: short, translucent fibers present (more than few)
E: long, translucent fiber present, and few, short translucent fibers present

TABLE 4
DOE Study Results ([ZnCl₂] = 7.0-15.0 mM, pH = 4.9-7.1)

| | | | | After 7 Days at 40° C. | | | | |
|---|---|---|---|---|---|---|---|---|
| | [TP508] | [ZnCl₂], | pH | | | Purity (area % | | |
| Entry | (mg/mL) | mM | (t₀) | pH | Appearance | vs control) | ≥10 μm | ≥25 μm |
| 76 | 150 | 7.0 | 5.0 | 4.9 | B | 78.0 | 740 | 64 |
| 77 | 150 | 7.0 | 5.0 | 4.9 | B | 81.2 | 276 | 22 |
| 78 | 150 | 7.0 | 6.0 | 6.0 | B | 59.7 | 262 | 26 |
| 79 | 150 | 7.0 | 6.0 | 5.9 | B | 68.5 | 306 | 22 |
| 80 | 150 | 7.0 | 7.0 | 7.1 | B | 36.2 | 694 | 48 |
| 81 | 150 | 7.0 | 7.0 | 6.8 | B | 43.3 | 256 | 24 |
| 82 | 150 | 11.0 | 5.0 | 4.9 | B | 86.8 | 214 | 20 |
| 83 | 150 | 11.0 | 5.0 | 4.9 | B | 86.1 | 258 | 22 |
| 84 | 150 | 11.0 | 6.0 | 5.9 | B | 81.1 | 246 | 52 |
| 85 | 150 | 11.0 | 6.0 | 5.9 | B | 90.1 | 156 | 24 |
| 86 | 150 | 11.0 | 7.0 | 7.0 | A | 57.5 | 238 | 24 |
| 87 | 150 | 11.0 | 6.0 | 6.0 | A | 85.0 | 174 | 16 |
| 88 | 150 | 11.0 | 7.0 | 7.1 | B | 52.3 | 252 | 50 |
| 89 | 150 | 11.0 | 7.0 | 7.0 | A | 62.9 | 274 | 78 |
| 90 | 150 | 15.0 | 5.0 | 5.0 | A | 87.9 | 276 | 120 |
| 91 | 150 | 15.0 | 5.0 | 4.9 | A | 87.0 | 94 | 8 |
| 92 | 150 | 15.0 | 6.0 | 5.9 | A | 89.4 | — | — |
| 93 | 150 | 15.0 | 6.0 | 5.9 | A | 84.1 | — | — |
| 94 | 150 | 15.0 | 7.0 | 6.8 | B | 74.6 | — | — |
| 95 | 150 | 15.0 | 7.0 | 6.7 | B | 80.8 | — | — |

A: clear, colorless liquid, no particles present
B: clear or slightly opalescent, colorless liquid, a few particles present
— = not performed

TABLE 5
DOE Peptide Monomer Response Surface for pH vs [ZnCl₂]

Peptide Response (Area %)
[ZnCl₂] (mM)

| | | 5.0 | 13.7 | 35.0 | 56.2 | 65.0 |
|---|---|---|---|---|---|---|
| pH (initial) | 5.5 | 3.2 | | | | 90.7 |
| | 5.9 | | 93.4 | | 92.4 | |
| | 7.0 | | | 94.3 | | |
| | 8.1 | | 64.2 | | 93.0 | |
| | 8.5 | 54.7 | | | | 91.6 |

TABLE 6
DOE Peptide Dimer Response Surface for pH vs [ZnCl₂]

Dimer Response (Area %)
[ZnCl₂] (mM)

| | | 5.0 | 13.7 | 35.0 | 56.2 | 65.0 |
|---|---|---|---|---|---|---|
| pH (initial) | 5.5 | 85.0 | | | | * |
| | 5.9 | | * | | * | |
| | 7.0 | | | * | | |
| | 8.1 | | 30.3 | | * | |
| | 8.5 | 40.2 | | | | 0.5 |

*Negligible amount of dimer versus control.

TABLE 7
DOE Peptide Monomer Response Surface for pH vs [ZnCl₂]

| | | Peptide Response (Area %) | | |
|---|---|---|---|---|
| | | [ZnCl₂] (mM) | | |
| | | 7.0 | 11.0 | 15.0 |
| pH (final) | 4.9-5.0 | 78.0, 81.2 | 86.8, 86.1 | 87.9, 87.0 |
| | 5.9-6.0 | 59.7, 68.5 | 81.1, 85.0, | 89.4, 84.1 |
| | | | 90.1 | |
| | 6.7-7.1 | 36.2, 43.3 | 57.5, 52.3, | 74.6, 80.8 |
| | | | 62.9 | |

1. A stable aqueous composition comprising 50 to 200 mg/mL TP508 or a TP508 derivative and ZnCl₂ or another suitable zinc salt at a concentration of 7 mM to 260 mM at a pH of 5 to 8.5.

2. The stable aqueous composition of claim 1, further comprising an anti-oxidant.

3. The stable aqueous composition of claim 2, wherein the anti-oxidant is selected from ascorbic acid, cysteine, methionine, monothioglycerol, sodium thiosulphate, sulfites, BHT, BHA, ascorbyl palmitate, propyl gallate, N-acetyl-L-cysteine (NAC), and Vitamin E.

4. The stable aqueous composition of claim 1, further comprising a sugar.

5. The stable aqueous composition of claim 4, wherein the sugar is selected from trehalose, glucose, or sucrose.

6. The stable aqueous composition of claim 1, further comprising a starch.

7. The stable aqueous composition of claim 6, wherein the starch is hydroxyethyl starch (HES).

8. The stable aqueous composition of claim 1, further comprising a sugar alcohol.

9. The stable aqueous composition of claim 8, wherein the sugar alcohol is mannitol and/or sorbitol.

10. The stable aqueous composition of claim 1, further comprising a chelator.

11. The stable aqueous composition of claim 10, wherein the chelator is selected from EDTA, tartaric acid, glycerin, and citric acid.

12. The stable aqueous composition of claim 1, further comprising a preservative.

13. The stable aqueous composition of claim 12, wherein the preservative is selected from benzyl alcohols, methyl parabens, propyl parabens, and mixtures thereof.

14. The stable aqueous composition of claim 1, further comprising additional excipients selected from an amino acid, surfactant, or a metal ion.

15. The stable aqueous composition of claim 14, wherein the amino acid is selected from histidine, proline, glycine, or methionine.


SEQUENCE LISTING (23 sequences)

SEQ ID NO: 1 - protein, 23 aa, synthetic construct
AGYKPDEGKRGDACEGDSGGPFV

SEQ ID NO: 2 - protein, 10 aa, synthetic construct
CEGDSGGPFV

SEQ ID NO: 3 - protein, 10 aa, synthetic construct
  VAR_SEQ 2: note = X is Glu or Gln
  VAR_SEQ 9: note = X is Phe, Met, Leu, His, or Val
CXGDSGGPXV

SEQ ID NO: 4 - protein, 14 aa, synthetic construct
  VAR_SEQ 6: note = X is Glu or Gln
  VAR_SEQ 13: note = X is Phe, Met, Leu, His or Val
RGDACXGDSGGPXV

SEQ ID NO: 5 - protein, 23 aa, synthetic construct
AGYKPDEGKRGDACEGDSGGPFV

SEQ ID NO: 6 - protein, 14 aa, synthetic construct
RGDACEGDSGGPFV

SEQ ID NO: 7 - protein, 33 aa, synthetic construct
DNMFCAGYKPDEGKRGDACEGDSGGPFVMKSPF

SEQ ID NO: 8 - protein, 23 aa, synthetic construct
  VAR_SEQ 15: note = X is Glu or Gln
  VAR_SEQ 22: note = X is Phe, Met, Leu, His or Val
AGYKPDEGKRGDACXGDSGGPXV

SEQ ID NO: 9 - protein, 33 aa, synthetic construct
  VAR_SEQ 20: note = X1 is Glu or Gln
  VAR_SEQ 27: note = X2 is Phe, Met, Leu, His or Val
DNMFCAGYKPDEGKRGDACXGDSGGPXVMKSPF

SEQ ID NO: 10 - protein, 14 aa, synthetic construct
  VAR_SEQ 5: note = X is alanine, glycine, serine or an S-protected cysteine
  VAR_SEQ 6: note = X is Glu or Gln
  VAR_SEQ 13: note = X is Phe, Met, Leu, His or Val
RGDAXXGDSGGPXV

SEQ ID NO: 11 - protein, 23 aa, synthetic construct
  VAR_SEQ 14: note = X is alanine, glycine, serine or an S-protected cysteine
AGYKPDEGKRGDAXEGDSGGPFV

SEQ ID NO: 12 - protein, 23 aa, synthetic construct
  VAR_SEQ 14: note = X is alanine, glycine, serine or an S-protected cysteine
  VAR_SEQ 15: note = X is Glu or Gln
  VAR_SEQ 22: note = X is Phe, Met, Leu, His or Val
AGYKPDEGKRGDAXXGDSGGPXV

SEQ ID NO: 13 - protein, 23 aa, synthetic construct
AGYKPDEGKRGDAAEGDSGGPFV

SEQ ID NO: 14 - protein, 23 aa, synthetic construct
AGYKPDEGKRGDAAEGDSGGPFV

SEQ ID NO: 15 - protein, 23 aa, synthetic construct
AGYKPDEGKRGDASEGDSGGPFV

SEQ ID NO: 16 - protein, 33 aa, synthetic construct
  VAR_SEQ 5: note = X is alanine, glycine, serine or an S-protected cysteine
  VAR_SEQ 19: note = X is alanine, glycine, serine or an S-protected cysteine
DNMFXAGYKPDEGKRGDAXEGDSGGPFVMKSPF

SEQ ID NO: 17 - protein, 34 aa, synthetic construct
  VAR_SEQ 5: note = X is alanine, glycine, serine or an S-protected cysteine
  VAR_SEQ 19: note = X is alanine, glycine, serine or an S-protected cysteine
  VAR_SEQ 20: note = X is Glu or Gln
  VAR_SEQ 28: note = X is Phe, Met, Leu, His or Val
DNMFXAGYKPDEGKRGDAXXGDASGGPXVMKSPF

SEQ ID NO: 18 - protein, 33 aa, synthetic construct
DNMFAAGYKPDEGKRGDAAEGDSGGPFVMDSPF

SEQ ID NO: 19 - protein, 33 aa, synthetic construct
DNMFAAGYKPDEGKRGDAAEGDSGGPFVMKSPF

SEQ ID NO: 20 - protein, 33 aa, synthetic construct
AGYKPDEGKRGDACEGDSGGPFVMKSPFNNRWY

SEQ ID NO: 21 - protein, 33 aa, synthetic construct
  VAR_SEQ 15: note = X is Glu Gln
  VAR_SEQ 22: note = X is Phe, Met, Leu, His or Val
AGYKPDEGKRGDACXGDSGGPXVMKSPFNNRWY

SEQ ID NO: 22 - protein, 33 aa, synthetic construct; identical to SEQ ID NO: 20

SEQ ID NO: 23 - protein, 33 aa, synthetic construct; identical to SEQ ID NO: 20